(19) United States
(12) Patent Application Publication
(10) Pub. No.: US 2025/0221355 A1
(43) Pub. Date: Jul. 10, 2025

(54) ENVIRONMENTAL MEMORY SEED AND USE THEREOF

(71) Applicant: KYUSHU UNIVERSITY, NATIONAL UNIVERSITY CORPORATION, Fukuoka-shi, Fukuoka (JP)

(72) Inventors: Yushi ISHIBASHI, Fukuoka-shi (JP); Chetphilin SURIYASAK, Fukuoka-shi (JP); Norimitsu HAMAOKA, Fukuoka-shi (JP)

(73) Assignee: KYUSHU UNIVERSITY, NATIONAL UNIVERSITY CORPORATION, Fukuoka-shi, Fukuoka (JP)

(21) Appl. No.: 18/852,581

(22) PCT Filed: Mar. 31, 2023
(86) PCT No.: PCT/JP2023/013510

§ 371 Date: Sep. 30, 2024

(30) Foreign Application Priority Data
Mar. 31, 2022 (JP) 2022-058473

(51) Int. Cl. A01H 3/00; A01H 5/10; A01H 6/46

(52) U.S. Cl. CPC A01H 3/00; A01H 5/10; A01H 6/4636

(57) ABSTRACT

The object is to clarify the relationship between environmental stress and seed quality, and to improve the yield and quality of crop seeds by a novel method different from the conventional genetic engineering methods. There is provided a seed of a plant having a promoter with an effectively controlled methylation level for any gene selected from the group consisting of the SLB1 gene, Hd1 gene, AGPS2b gene, GBSSI gene, SuSy2 gene, AMy1A gene, and Amy3D gene or a product thereof, or a harvested product obtained therefrom or a processed product thereof. There is also provided a method for producing a seed of a plant having a useful characteristic, which comprises: the step of cultivating a first generation plant under an environmental stress; and the step of obtaining a seed with environmental stress memory from the cultivated plant or a progeny thereof.

### TECHNICAL FIELD

The present invention relates to environmental memory seeds and use thereof. The present invention is useful in the fields of plant improvement, agricultural crop production, industrial production of agricultural crops, and so forth.

### BACKGROUND ART

Recent food problems caused by global environmental change and population growth trends have reminded us that improving crop yields is an important global issue. Environmental stresses such as high temperatures, drought, and salinity can cause losses in crop production. In particular, the ambient temperature at which crops are cultivated strongly affects growth and development during both the vegetative and reproductive seasons. Various studies have been conducted on the effects of environmental stresses on crops and methods to improve environmental stress tolerance in crops.

For example, Patent document 1 proposes a method for treating seeds, which comprises the step of treating seeds for sowing with UV-B irradiation, and describes that this method is for improving the yield of harvestable crop material. In the section of Examples, lettuce and kale obtained from UV-B irradiated seeds for sowing are analyzed for flavonoid content, drought stress tolerance, seedling size, etc. This reference also states that the action of UV-B concerning seed treatment relates to mutations at the UVR8 locus and relating to UVR8 activity. Patent document 2 proposes a method for improving germination rate of buried seeds in topsoil, which comprises the stress application step. Further, Patent document 3 proposes a method for early flowering by seed reproduction of rosaceous plants belonging to a group of plants called stone fruits, which comprises the step of performing a stress treatment to seedlings to promote flower bud differentiation. Furthermore, Patent document 4 describes a method for enhancing plant characteristics, which comprises a freezing step for freezing a plant tissue, a thawing step for thawing the frozen plant tissue, and a generation step for generating a plant from the thawed plant tissue. However, these references do not describe the effects of stress treatment on plants of the next generation.

Patent document 5 proposes a method for enhancing stress resistance in the next generation of a plant characterized by comprising the step of subjecting the current generation plant to a stress treatment during at least the nutrient growth period. In the section of Examples of this reference, it is described that the rice cultivars Hitomebore and Sasanishiki were subjected to salinity or low light stress during the nutrient growth period to obtain seeds, and the cold tolerance during the boot stage was enhanced in the next generation of rice cultivated from the seeds. Patent document 6 proposes a method for controlling flowering time in the next generation of a plant, which comprises the step of applying a stress treatment during the nutritional growth period of the current generation of the plant. In the section of Examples of this reference, it is confirmed that the next generations of the rice cultivars Hitomebore and Sasanishiki, grown from seeds harvested from the cultivars grown in a low temperature environment showed earlier heading than the next generation grown from seeds harvested from the cultivars grown in a high temperature environment. It was also stated that, as a part of the reasons for this, involvement of water temperature during the nutrient growth period in the control of the heading of the next generation is suggested, suggesting that temperature differences during the historical period are memorized by the next generation.

Recently, it has become clear that plants memorize past environmental events and use this memory to help them respond to recurrent environmental events. Epigenetic mechanisms utilized by plants under various environmental stresses have been elucidated and are known to play an important role in the regulation of gene expression via small RNAs, histone modifications, DNA methylation, etc. (Non-patent document 1). The mechanism of delayed seed germination in rice (Oryza sativa L.) due to high temperature during the grain filling stage has been studied, and it has been reported that high temperature during grain filling causes DNA methylation at the promoters of abscisic acid (ABA) catabolism-related genes and a-amylase gene (Non-patent document 2).

### PRIOR ART REFERENCES

### Patent Documents

Patent document 1: Japanese Patent Publication (Kohyo) No. 2017-529076
Patent document 2: Japanese Patent Publication (Kokai) No. 2009-82022
Patent document 3: Japanese Patent Publication (Kokai) No. 2008-48664
Patent document 4: Japanese Patent Publication (Kokai) No. 2018-183112
Patent document 5: Japanese Patent Publication (Kokai) No. 2015-181370
Patent document 6: Japanese Patent Publication (Kokai) No. 2016-182094

### Non-Patent Documents

Non-patent document 1: Kinoshita, T. et al., Epigenetic Memory for Stress Response and Adaptation in Plants. Plant Cell Physiol. 55(11): 1859-1863 (2014)
Non-patent document 2: Suriyasak, C. et al., Mechanism of delayed seed germination caused by high temperature during grain filling in rice (Oryza sativa L.). Scientific Reports, 10:17378 (2020)

### SUMMARY OF THE INVENTION

### Object to be Achieved by the Invention

The inventors of the present invention have promoted researches focusing on the fruit (seed), which is an important trait in crop yield and quality. In particular, the relationship between seed quality and grain filling environment has been studied because environmental factors during the grain filling stage strongly influence the factors that reduce seed quality, such as high temperature injury in paddy rice. If the traits involved in modifying seed weight and the relationship between the grain filling environment and seed quality are clarified, it will become possible to improve yield and quality of crop seeds by novel methods instead of the conventional genetic engineering methods.

### Means for Achieving the Object

The inventors of the present invention found that if rice plants are cultivated from seeds obtained from rice plants cultivated under heat stress during the grain filling stage, delayed germination, increased biomass, accelerated flowering, increased seed yield, and heat tolerance during grain filling are conferred. The inventors of the present invention also found that seeds memorize such environmental stresses, and thus seeds for crop production with enhanced environmental stress tolerance can be produced, and accomplished the present invention.

The present invention provides the followings.

[1] A seed of a plant or a product thereof, or a harvested product obtained therefrom or a processed product thereof, the seed having a promoter with an effectively controlled methylation level, the promoter being for any gene selected from the group consisting of the SLB1 gene, Hd1 gene, AGPS2b gene, GBSSI gene, SuSy2 gene, AMy1A gene, and Amy3D gene.
[2] The seed or a product thereof, or a harvested product obtained therefrom or a processed product thereof according to 1, wherein the promoter is a hypermethylated promoter for any gene selected from the group consisting of the SLB1 gene, AGPS2b gene, GBSSI gene, and SuSy2 gene.
[3] The seed or a product thereof, or a harvested product obtained therefrom or a processed product thereof according to 1 or 2, wherein the promoter is a hypomethylated promoter for any gene selected from the group consisting of the Hd1 gene, AMy1A gene, and Amy3D gene.
[4] The seed or a product thereof, or a harvested product obtained therefrom or a processed product thereof according to any one of 1 to 3, wherein the promoter is the SLB1 gene P2 promoter.
[5] The seed or a product thereof, or a harvested product obtained therefrom or a processed product thereof according to 4, wherein the plant is a Poaceae plant, and the promoter has a relative methylation level of 1.6% input or higher.
[6] A method for producing a seed of a plant having a useful characteristic, which comprises:
the step of cultivating a first generation plant under an environmental stress; and the step of obtaining a seed with environmental stress memory from the cultivated plant or a progeny thereof.
   [7] The production method according to 6, wherein the environmental stress memory is a change in methylation level of DNA in the promoter region of the Heading date 1 (Hd1) gene.
   [8] The production method according to 6 or 7, wherein the environmental stress is temperature stress or low nitrogen stress during the seed developing period. [9] The production method according to any one of 6 to 8, wherein the plant is a Poaceae plant, and the useful characteristic is any selected from the group consisting of increased biomass, accelerated flowering, increased seed yield, acquired heat tolerance during grain filling, acquired low nitrogen tolerance, and early growth.
   [10] The production method according to any one of 6 to 9, wherein the plant is a Poaceae plant, and the useful characteristic is any selected from the group consisting of increased tiller number, increased yield per unit area, earlier heading stage, and low chalky grain rate.
   [11] The production method according to any one of 6 to 8, wherein the plant is a plant belonging to the family Poaceae, Fabaceae, or Asteraceae, and the environmental stress is temperature stress during the seed developing period.
   [12] The production method according to any one of 6 to 11, wherein the plant is rice, the environmental stress is heat stress during the seed developing period, and the heat stress is cultivating the plant at an environmental temperature higher by 5° C. or more than the average temperature during that period for 6 weeks or longer after heading.

The present invention also provides the followings.

[1] A seed of a plant having a promoter with an effectively controlled methylation level for any gene selected from the group consisting of the SLB1 gene, Hd1 gene, AGPS2b gene, GBSSI gene, SuSy2 gene, AMy1A gene, and Amy3D gene or a product thereof, or a harvested product obtained therefrom or a processed product thereof.
[2] The seed or a product thereof, or a harvested product obtained therefrom or a processed product thereof according to 1, wherein the promoter is a hypermethylated promoter for any gene selected from the group consisting of the SLB1 gene, AGPS2b gene, GBSSI gene, and SuSy2 gene.
[3] The seed or a product thereof, or a harvested product obtained therefrom or a processed product thereof according to 1, wherein the promoter is a hypomethylated promoter for any gene selected from the group consisting of the Hd1 gene, AMy1A gene, and Amy3D gene.
[4] The seed or a product thereof, or a harvested product obtained therefrom or a processed product thereof according to 2, wherein the promoter is the SLB1 gene P2 promoter.
[5] The seed or a product thereof, or a harvested product obtained therefrom or a processed product thereof according to 4, wherein the plant is a Poaceae plant, and the promoter has a relative methylation level of 1.6% input or higher.
[6] A method for producing a seed of a plant having a useful characteristic, which comprises:
the step of cultivating a first generation plant under an environmental stress; and the step of obtaining a seed with environmental stress memory from the grown plant or a progeny thereof
   [7] The production method according to 6, wherein the environmental stress memory is a change in methylation level of DNA in the promoter region of the Heading date 1 (Hd1) gene.
   [8] The production method according to 6 or 7, wherein the environmental stress is heat stress or low nitrogen stress during the seed developing period.
   [9] The production method according to any one of 6 to 8, wherein the plant is a Poaceae plant, and the useful characteristic is any selected from the group consisting of increased biomass, accelerated flowering, increased seed yield, and acquired heat tolerance during grain filling.
   [10] The production method according to any one of 6 to 9, wherein the plant is a Poaceae plant, and the useful characteristic is any selected from the group consisting of increased tiller number, increased yield per unit area, earlier heading stage, and low chalky grain rate.
   [11] The production method according to any one of 6 to 10, wherein the plant is a Poaceae plant, the environmental stress is heat stress during the seed developing period, and the heat stress is cultivating the plant at an environmental temperature higher by 5° C. or more than the average temperature during that period for 6 weeks or longer after heading.

### Effects of the Invention

Seeds for high-yielding and early maturing agricultural crop production can be obtained by using existing good varieties without genetic manipulation and without breeding by crossbreeding or fixation. Seeds with environmental stress memory can contribute to stable production of agricultural crops under global environmental changes such as global warming.

Seeds for agricultural crops crop production with enhanced environmental stress tolerance can be produced.

Use of the present invention for the production of high-yielding and early maturing agricultural crops, production of seeds for factory production, production of agricultural crops in factories, and construction of agricultural crop and seed production system can be expected.

### BRIEF DESCRIPTION OF THE DRAWINGS

FIG. 1 Effect of heat stress during grain filling on the next generation plant. a, Phenotypes of the control group and heat stressed-progeny plants grown under natural condition. b and c, Tiller number and plant height (n=8) at maximum tillering stage (55 days after sowing, DAS). d, Morphology of flag leaves and longitudinal sections thereof stained with 0.2% toluidine blue. e and f, Flag leaf length (n=50) and thickness (n=10). g, Early heading phenotype of heat stressed plants under natural condition (82 DAS). h, Distribution of heading rates with average heading dates of total 200 individual plants in twenty pots for each of the control group and heat stress group. i, Yields of brown rice harvested from single control and heat-stressed plant (calculated at 15% water content, n=8). Significant differences in student's t-test are shown with **(P<0.01) and ***(P<0.001).

FIG. 2 Global genome-wide methylation levels analyzed by whole genome bisulfite sequencing of the control seeds and heat-developed seeds (seeds subjected to heat stress during the grain filling stage). a, Chromosome maps of average methylation levels of different methylation contexts (CG, CHG, and CHH) at 1 Mbp window size (CS, control seeds; HDS, heat-developed seeds). b, Distributions of hypermethylated and hypo-methylated DMRs of the contexts for different genomic regions. c, Gene ontology analysis of all DMRs.

FIG. 3 Heat stress during grain filling induces transcriptional changes of progeny plants at developmental stage. a, Total differentially expressed genes (DEGs) obtained by RNA sequencing of developing leaves at 55 DAS, and b, GO analysis of total DEGs. c and d, Transcriptional levels of OsSLB1 in root and leaf (relative expressions to expressions of OsActin and OsUBQ, respectively) (CS, control seeds; HDS, heat-developed seeds). e, Heatmap showing expression levels of heading related genes in rice. f and g, Relative expressions of OsHd1 and OsHd3a prior to anthesis normalized to OsUBQ. Error bars represent standard deviations of three biological replicates. Significant differences in student's t-test are shown with *(P<0.05) and **(P<0.01).

FIG. 4 Methylation levels of OsSLB1, OsHd1 and OsHd3a promoters determined by MeDIP-qPCR in the control and heat stressed seeds and developing organs. a, Promoter regions. b, c, and d, Methylation levels of OsSLB1, OsHd1, and OsHd3a promoters in seeds. e, Methylation levels of OsSLB1 promoter in roots at the maximum tillering stage. f, and g, Methylation levels of OsHd1 and OsHd3a promoters in leaves of 55 DAS. Significant differences according to Student's t-test are indicated with *(p<0.05) and **(p<0.01) (n=4).

FIG. 5 Heat stress during grain frilling induces thermotolerance of progeny plants. a, Expression of starch synthesis genes (OsAGPS2b, OsGBSSI, and OsSuSy2) and starch degrading α-amylase genes (OsAmy1A, and OsAmy3D) in the seeds during grain filling. b, Flag leaf SPAD values measured with 5-day interval after anthesis. c, Photosynthetic rates at 26 DAF (days after flowering) in flag leaves. d, Percentages and distribution of different types of chalkiness in the harvested brown rice. Relative expression levels were compared with OsActin. Different alphabets represent significant differences at the 5% probability level according to Tukey's test (SPAD and photosynthetic rate, n=4; gene expression, n=3).

FIG. 6 Methylation levels of promoters of starch synthesis and starch degradation genes determined by MeDIP-qPCR in the control and heat stressed seeds and developing organs. a, and b, Methylation levels of starch synthesis and starch degradation gene promoters in seeds and developing grains at 15 DAF. Significant differences according to Student's t-test are indicated with *(p<0.05) and **(p<0.01), or different alphabets represent significant differences at 5% probability level according to Tukey's test (n=4).

FIG. 7 Heat stress during grain filling for wheat (germination and biomass).

FIG. 8 Heat stress during grain filling for wheat (heading and yield)

FIG. 9 Biomass increase in rice with heat stress memory under low nitrogen condition.

FIG. 10 Heat tolerance of rice with heat stress memory.

FIG. 11 Effect of heat stress memory in seeds on biomass of barley (Hordeum vulgare (Barley) cv. Ichibanboshi).

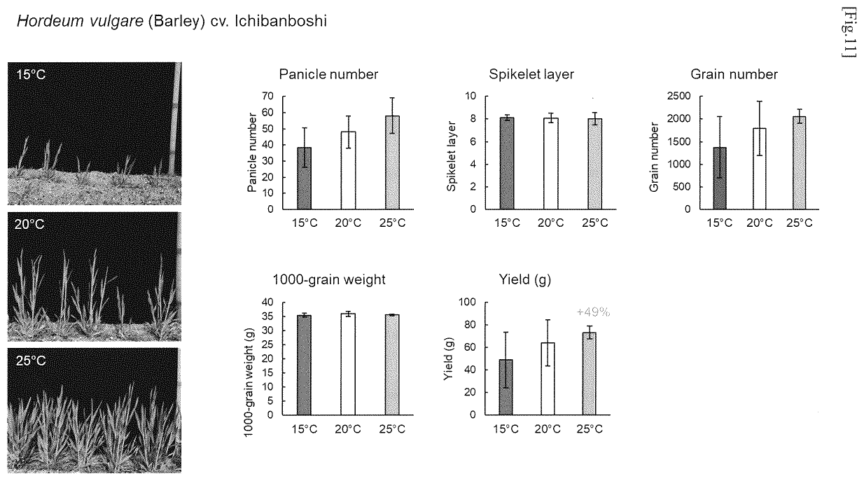

FIG. 12 Effect of heat stress memory in seeds on biomass of lettuce (Lactusa sativa).

FIG. 13 Effect of heat stress memory in seeds on biomass of cowpea (Vigna unguiculata (Cowpea) cv. IT98K-205-8).

FIG. 14 Effect of heat stress memory in seeds on biomass of soybean (Glycine Max (L.) Merr. (Soybean) cv. Fukuyutaka).

FIG. 15 Sequences of the promoters.

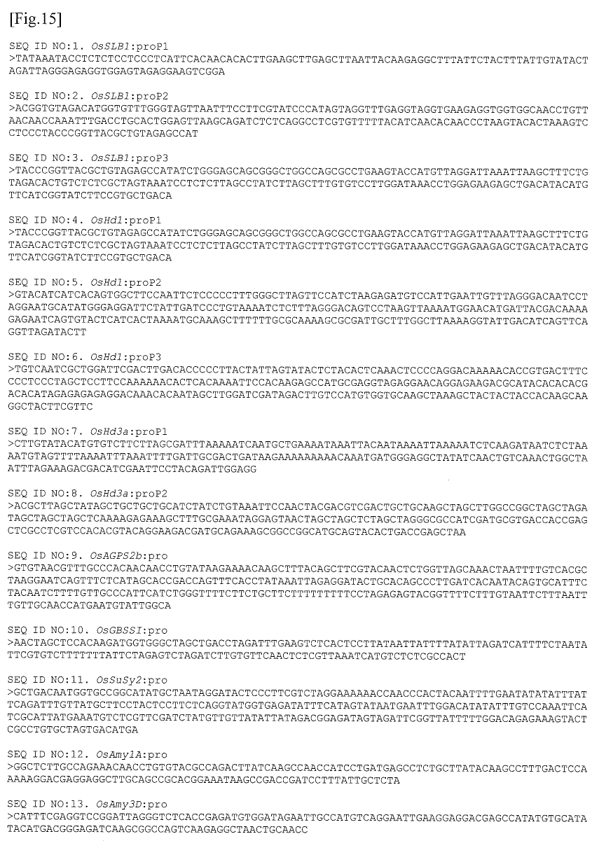

### MODES FOR CARRYING OUT THE INVENTION

### Environmental Memory Seed

The present invention provides an environmental memory seed or a product thereof, or a harvested product obtained therefrom or a processed product thereof. Concerning the present invention, explanations may be made for the seed among the seed, a product thereof, a harvested product obtained therefrom, and a processed product thereof, but such explanations will be appropriately applied to the product thereof, harvested product obtained therefrom, and processed product thereof and understood by those skilled in the art.

One example of the environmental memory seed is a seed having a polynucleotide with an effectively regulated methylation level. Examples of such a polynucleotide include a promoter of any gene selected from the group consisting of the SLB1 gene, Hd1 gene, AGPS2b gene, GBSSI gene, SuSy2 gene, AMy1A gene, and Amy3D gene.

In one of the preferred embodiments, the promoter is a promoter of any gene selected from the group consisting of the SLB1 gene, AGPS2b gene, GBSSI gene, and SuSy2 gene, and the effectively regulated methylation level of the promoter is a methylation level different from (higher or lower than) the methylation level of the reference seed, and is high or low enough to alter expression of a downstream gene from that of the reference seed.

In another embodiment, the promoter is a promoter of any gene selected from the group consisting of the Hd1 gene, AMy1A gene, and Amy3D gene, and the methylation level of the promoter is preferably low enough to promote expression of a downstream gene.

In one particularly preferred embodiment, the promoter with a controlled methylation level is the SLB1 gene P2 promoter. In this case, if the plant is a Poaceae plant (preferably rice), the methylation level is preferably 1.6% input or higher in terms of relative methylation level.

### [Promoter]

The promoter relating to the present invention will be explained below by referring to Oryza sativa L. cv. Nipponbare as an example.

In one of the preferred embodiments, the P1 promoter of the SLB1 gene (SLB1: proP1) refers to any of the followings:
(A) a polynucleotide consisting of the sequence of SEQ ID NO: 1; and
(B) a polynucleotide consisting of a sequence having a high sequence identity to the sequence of SEQ ID NO: 1 and having an activity to regulate expression of the SLB1 gene.

In one of the preferred embodiments, the P2 promoter of the SLB1 gene (SLB1: proP2) refers to any of the followings:
(A) a polynucleotide consisting of the sequence of SEQ ID NO: 2; and
(B) a polynucleotide consisting of a sequence having a high sequence identity to the sequence of SEQ ID NO: 2 and having an activity to regulate expression of the SLB1 gene.

In one of the preferred embodiments, the P3 promoter of the SLB1 gene (SLB1: proP3) refers to any of the followings:
(A) a polynucleotide consisting of the sequence of SEQ ID NO: 3; and
(B) a polynucleotide consisting of a sequence having a high sequence identity to the sequence of SEQ ID NO: 3 and having an activity to regulate expression of the SLB1 gene.

In one of the preferred embodiments, the P1 promoter of the Hd1 gene (Hd1: proP1) refers to any of the followings:
(A) a polynucleotide consisting of the sequence of SEQ ID NO: 4; and
(B) a polynucleotide consisting of a sequence having a high sequence identity to the sequence of SEQ ID NO: 4 and having an activity to regulate expression of the Hd1 gene.

In one of the preferred embodiments, the P2 promoter of the Hd1 gene (Hd1: proP2) refers to any of the followings:
(A) a polynucleotide consisting of the sequence of SEQ ID NO: 5; and
(B) a polynucleotide consisting of a sequence having a high sequence identity to the sequence of SEQ ID NO: 5 and having an activity to regulate expression of the Hd1 gene.

In one of the preferred embodiments, the P3 promoter of the Hd1 gene (Hd1: proP3) refers to any of the followings:
(A) a polynucleotide consisting of the sequence of SEQ ID NO: 6; and
(B) a polynucleotide consisting of a sequence having a high sequence identity to the sequence of SEQ ID NO: 6 and having an activity to regulate expression of the Hd1 gene.

In one of the preferred embodiments, the P1 promoter of the Hd3a gene (Hd3a: pro1) refers to any of the followings:
(A) a polynucleotide consisting of the sequence of SEQ ID NO: 7; and
(B) a polynucleotide consisting of a sequence having a high sequence identity to the sequence of SEQ ID NO: 7 and having an activity to regulate expression of the Hd3a gene.

In one of the preferred embodiments, the P2 promoter of the Hd3a gene (Hd3a: pro2) refers to any of the followings:
(A) a polynucleotide consisting of the sequence of SEQ ID NO: 8; and
(B) a polynucleotide consisting of a sequence having a high sequence identity to the sequence of SEQ ID NO: 8 and having an activity to regulate expression of the Hd3a gene.

In one of the preferred embodiments, the promoter of the AGPS2b gene (AGPS2b: pro) refers to any of the followings:
(A) a polynucleotide consisting of the sequence of SEQ ID NO: 9; and
(B) a polynucleotide consisting of a sequence having a high sequence identity to the sequence of SEQ ID NO: 9 and having an activity to regulate expression of the AGPS2b gene.

In one of the preferred embodiments, the promoter of the GBSSI gene (GBSSI: pro) refers to any of the followings:
(A) a polynucleotide consisting of the sequence of SEQ ID NO: 10; and
(B) a polynucleotide consisting of a sequence having a high sequence identity to the sequence of SEQ ID NO: 10 and having an activity to regulate expression of the GBSSI gene.

In one of the preferred embodiments, the promoter of the SuSy2 gene (SuSy2: pro) refers to any of the followings:
(A) a polynucleotide consisting of the sequence of SEQ ID NO: 11; and
(B) a polynucleotide consisting of a sequence having a high sequence identity to the sequence of SEQ ID NO: 11 and having an activity to regulate expression of the SuSy2 gene.

In one of the preferred embodiments, the promoter of the Amy1A gene (Amy1A: pro) refers to any of the followings:
(A) a polynucleotide consisting of the sequence of SEQ ID NO: 12; and
(B) a polynucleotide consisting of a sequence having a high sequence identity to the sequence of SEQ ID NO: 12 and having an activity to regulate expression of the Amy1A gene.

In one of the preferred embodiments, the promoter of the AGPS2b gene (AGPS2b: pro) refers to any of the followings:
(A) a polynucleotide consisting of the sequence of SEQ ID NO: 13; and
(B) a polynucleotide consisting of a sequence having a high sequence identity to the sequence of SEQ ID NO: 13 and having an activity to regulate expression of the AGPS2b gene.

The sequences of SEQ ID NOS: 1 to 13 mentioned in the sequence listing, a part of this description, are sequences derived from rice (Oryza sativa L.).

Other than rice, these genes and their promoters are also shared by wheat, barley, lettuce, cowpea, and soybean. ID numbers of the related sequences in the Phytozome (https://phytozome-next.jgi.doe.gov/) database are listed in the following table. The related sequences can be obtained by those skilled in the art and used in place of the sequences of SEQ ID NOS: 1 to 13 mentioned above, as required.

TABLE 1

| Genes | Soybean | Lettuce | Cowpea | Wheat | Barley |
|---|---|---|---|---|---|
| SLB1 | Glyma.04G052100.1 | Lsat_1_v5_gn_6_14580.1 | Vigun08g039400.1 | Traes_3AL_8BD2EAD06.1 | HORVU4Hr1G079620.3 |
| Hd1 | Glyma.08G255200.1 | Lsat_1_v5_gn_2_86121.1 | Vigun05g024400.1 | Traes_7BS_E337E82FB.1 | HORVU7Hr1G043030.10 |
| AGPS2b | Glyma.14G009300.1 | Lsat_1_v5_gn_7_98980.1 | Vigun08g214900.1 | Traes_7AS_1B2A8C929.2 | HORVU7Hr1G067620.2 |
| GBSSI | Glyma.20G218100.1 | Lsat_1_v5_gn_2_73301.1 | Vigun07g163500.1 | Traes_4AL_4B9D56131.1 | HORVU7Hr1G012380.5 |
| SuSy2 | Glyma.15G151000.1 | Lsat_1_v5_gn_5_119340.1 | Vigun09g041800.1 | Traes_7DS_529BAB150.2 | HORVU7Hr1G033230.8 |
| Amy1A | Glyma.17G242400.1 | Lsat_1_v5_gn_6_112060.1 | Vigun10g049800.1 | Traes_6AL_AC7FC445B.1 | HORVU6Hr1G080790.1 H |
| Amy3D | Glyma.17G242400.1 | Lsat_1_v5_gn_6_112060.1 | Vigun10g049800.1 | Traes_5BL_C3DA62711.1 | HORVU5Hr1G068350.1 H |

### [Nucleotide Sequence Identity]

For the present invention, the term “sequence identity” referred to for nucleotide sequence (also referred to as base sequence) means percentage of the number of matching nucleotides shared between two sequences aligned in an optimal manner, unless especially noted. That is, it can be calculated in accordance with the following equation: Identity=(Number of matched positions/Total number of positions)×100. This can be calculated by using commercially available algorithms. Such algorithms are also incorporated in the NBLAST and XBLAST programs described in Altschul et al. J. Mol. Biol. 215 (1990) 403-410. The calculation of nucleotide sequence identity can be performed by using programs known to those skilled in the art (e.g., BLASTN, BLASTP, BLASTX, and ClustalW). When using such programs, the parameters can be appropriately set by those skilled in the art, or the default parameters of each program may be used. The specific procedures of these analysis methods are also well known to those skilled in the art.

For the present invention, the term high identity referred to for a sequence means a sequence identity of 64% or higher, 65% or higher, 69% or higher, 70% or higher, 72% or higher, 75% or higher, 76% or higher, 77% or higher, 78% or higher, or 79% or higher, preferably 80% or higher, 81% or higher, 83% or higher, or 84% or higher, more preferably 85% or higher, 86% or higher, or 89% or higher, further preferably 90% or higher, further preferably 95% or higher, further preferably 97.5% or higher, further preferably 99% or higher, in all cases, unless especially noted.

The sequence identities between the promoter regions of the genes derived from rice (regions of the 3000 base length portion upstream of each gene) and corresponding regions of other plants are as follows.

1. SLB1 gene

### 1.1 Wheat:

Gene ID: Traes_3AL_8BD2EAD06.1 (Thromboxane-A synthase, CYTOCHROME P450 711A1)
Sequence identity: 75%

### 1.2 Maize:

Gene ID: Zm00001d039697_T001 (Thromboxane-A synthase, CYTOCHROME P450 711A1)
Sequence identity: 72%

### 1.3 Lettuce:

Gene ID: Lsat_1_v5_gn_6_14580.1 (Thromboxane-A synthase, CYTOCHROME P450 B-class)
Sequence identity: 70%
1.4 Brassica rapa:
Gene ID: Brara.C02476.1 (Thromboxane-A synthase, CYTOCHROME P450 B-class)
Sequence identity: 69%
2. Hd1 gene

### 2.1 Wheat:

Gene ID: Traes_7DS_46E811D74.1 (CCT-motif B-box Zinc finger)
Sequence identity: 81%

### 2.2 Maize:

Gene ID: Zm00001d045735_T002 (CCT-motif B-box Zinc finger)
Sequence identity: 72%

### 2.3 Lettuce:

Gene ID: Lsat_1_v5_gn_2_86121.1 (BBOX Zinc finger protein CONSTANS related) Sequence identity: 70%
2.4 Brassica rapa:

### Gene ID: Brara.A02054.1 (ZINC FINGER PROTEIN CONSTANS-RELATED)

Sequence identity: 83%
3. Hd3a gene

### 3.1 Wheat:

Gene ID: Traes_7AS_EBD5F1F54.1 (Flowering locus T-related)
Sequence identity: 86%

### 3.2 Maize:

Gene ID: Zm00001d036242_T001 (Flowering locus T-related)
Sequence identity: 89%

### 3.3 Lettuce:

Gene ID: Lsat_1_v5_gn_2_17881.1 (Flowering locus T)
Sequence identity: 78%
3.4 Turnip mustard:
Gene ID: Brara.G03476.1 (Flowering locus-T)
Sequence identity: 69%
4. AGPS2b gene

### 4.1 Wheat:

Gene ID: Traes 7AS_1B2A8C929.2 (Glucose-1-phosphate adenylyltransferase/ADP-glucose synthase)
Sequence identity: 90%

### 4.2 Maize:

Gene ID: Zm00001d025723_T001 (Glucose-1-phosphate adenylyltransferase/ADP-glucose synthase)
Sequence identity: 72%

### 4.3 Lettuce:

### Gene ID: Lsat_1_v5_gn_7_98980.1 (GLUCOSE-1-PHOSPHATE ADENYLYLTRANSFERASE SMALL SUBUNIT, CHLOROPLASTIC)

Sequence identity: 79%
4.4 Brassica rapa:

### Gene ID: Brara.G00430.1 (GLUCOSE-1-PHOSPHATE ADENYLYLTRANSFERASE SMALL SUBUNIT, CHLOROPLASTIC)

Sequence identity: 79%
5. GBSSI gene

### 5.1 Wheat:

Gene ID: Traes_4AL_4B9D56131.1 (NDP-glucose--starch glucosyltransferase/Waxy protein)
Sequence identity: 78%

### 5.2 Maize:

Gene ID: Zm00001d045462_T001 (NDP-glucose--starch glucosyltransferase/Waxy protein)
Sequence identity: 90%

### 5.3 Lettuce:

Gene ID: Lsat_1_v5_gn_2_73301.1 (NDP-glucose--starch glucosyltransferase/Waxy protein)
Sequence identity: 76%
5.4 Brassica rapa:
Gene ID: Brara.E01764.1 (NDP-glucose--starch glucosyltransferase/Waxy protein)
Sequence identity: 72%
6. SuSy2 gene

### 6.1 Wheat:

### Gene ID: Traes_7DS_529BAB150.2 (GLYCOSYLTRANSFERASE)

Sequence identity: 81%

### 6.2 Maize:

### Gene ID: Zm00001d045042_T014 (SUCROSE SYNTHASE)

Sequence identity: 84%

### 6.3 Lettuce:

### Gene ID: Lsat_1_v5_gn_5_119340.1 (GLYCOSYLTRANSFERASE//SUCROSE SYNTHASE 1-RELATED)

Sequence identity: 77%
6.4 Brassica rapa:
Gene ID: Brara.C00954.1 (Sucrose synthase)
Sequence identity: 70%
7. Amy1A gene

### 7.1 Wheat:

### Gene ID: Traes 6AL_AC7FC445B.1 (ALPHA-AMYLASE)

Sequence identity: 81%

### 7.2 Maize:

### Gene ID: Zm00001d018159_T001 (ALPHA-AMYLASE)

Sequence identity: 72%

### 7.3 Lettuce:

### Gene ID: Lsat_1_v5_gn_6_112060.1 L (ALPHA-AMYLASE)

Sequence identity: 64%
7.4 Brassica rapa:

### Gene ID: Brara.A02054.1 (ALPHA-AMYLASE)

Sequence identity: 65%
8. Amy3D gene

### 8.1 Wheat:

### Gene ID: Traes 5BL_C3DA62711.1 (ALPHA-AMYLASE)

Sequence identity: 81%

### 8.2 Maize:

### Gene ID: Zm00001d031794_T001 (ALPHA-AMYLASE)

Sequence identity: 89%

### 8.3 Lettuce:

Gene ID: at_1_v5_gn_6_112060.1 (ALPHA-AMYLASE)
Sequence identity: 70%
8.4 Brassica rapa:

### Gene ID: Brara.A02054.1 (ALPHA-AMYLASE)

Sequence identity: 70%

### [Methylation Level]

For the present invention, the expression that the methylation level is effectively controlled means that the promoter is methylated to such an extent that it can control expression of a downstream gene, unless especially noted. The degree of methylation can also be higher or lower by a certain rate compared with methylation level of a standard individual, if any.

The methylation level can be determined by measuring the degree of methylation for DNA extracted from the target seed by an appropriate method. The measurement method is not particularly limited, and there are (A) a method of analyzing methylated DNA after fragmenting and concentrating it, (B) an analysis method of subjecting methylated DNA to a bisulfite treatment and sequencing it, (C) an analysis method using a methylation-sensitive restriction enzyme, (D) an analysis method using a methylation-specific PCR method, and so forth, any of which may be used.

One preferred example is a method of fragmenting single-stranded DNA (ssDNA) by sonication or with an enzyme, and concentrating 5-methylcytosine (5-mC) on single-stranded DNA fragments by immunoprecipitation using an antibody directed to 5-mC. For this measurement, commercially available kits, e.g., MeDIP Kit, can be used.

The methylation level can be expressed in terms of % input as the ratio of methylated DNA to DNA used for the analysis (input) (https://www.diagenode.com/files/products/kits/magmedip-qpcr-kit-manual.pdf).

Examples of the promoter and preferred methylation level are shown below.

P1 promoter of SLB1 gene
Relative methylation level: 0.028% input or higher, preferably 0.046% input or higher, more preferably 0.049% input or higher, further preferably 0.062% input or higher.
P2 promoter of SLB1 gene
Relative methylation level: 0.116% input or higher, preferably 0.148% input or higher, more preferably 0.195% input or higher, further preferably 0.198% input or higher.
P3 promoter of SLB1 gene
Relative methylation level: 0.144% input or higher, preferably 0.163% input or higher, more preferably 0.169% input or higher, further preferably 0.171% input or higher.
P1 promoter of Hd1 gene
Relative methylation level: 0.222% input or higher, preferably 0.308% input or higher, more preferably 0.558% input or higher, further preferably 0.761% input or higher.
P2 promoter of Hd1 gene
Relative methylation level: 0.019% input or higher, preferably 0.032% input or higher, more preferably 0.040% input or higher, further preferably 0.049% input or higher.
P3 promoter of Hd1 gene
Relative methylation level: 0.002% input or higher, preferably 0.004% input or higher, more preferably 0.008% input or higher, further preferably 0.012% input or higher.
P1 promoter of Hd3 gene
Relative methylation level: 0.009% input or higher, preferably 0.011% input or higher, more preferably 0.012% input or higher, further preferably 0.015% input or higher.
P2 promoter of Hd3 gene
Relative methylation level: 0.002% input or higher, preferably 0.005% input or higher, more preferably 0.006% input or higher, further preferably 0.012% input or higher.
Promoter of AGPS2b gene
Relative methylation level: 0.003% input or higher, preferably 0.010% input or higher, more preferably 0.018% input or higher, further preferably 0.022% input or higher.
Promoter of GBSSI gene
Relative methylation level: 0.019% input or higher, preferably 0.027% input or higher, more preferably 0.036% input or higher, further preferably 0.040% input or higher.
Promoter of SuSy2 gene
Relative methylation level: 0.005% input or higher, preferably 0.010% input or higher, more preferably 0.014% input or higher, further preferably 0.019% input or higher.
Promoter of AMy1A gene
Relative methylation level: 0.027% input or higher, preferably 0.033% input or higher, more preferably 0.039% input or higher, further preferably 0.065% input or higher.
P3 promoter of Amy3D gene
Relative methylation level: 0.005% input or more, preferably 0.006% input or higher, more preferably 0.007% input or higher, further preferably 0.009% input or higher.

### [Plant]

The present invention can be applied to a variety of plants. Examples of the plant include those belonging to any of the families selected from Poaceae, Asteraceae, Lamiaceae, Brassicaceae, Apiaceae, and Amaranthaceae.

The plant to which the present invention is applied is not particularly limited, but examples of plants to which the present invention can be preferably applied include plants belonging to the class Monocotyledonae (monocotyledons), more preferably plants belonging to the subclass Commelinidae, further preferably plants belonging to the family Poaceae (Gramineae), of which examples are plants belonging to any of the genera Oryza, Hordeum, Secale, Triticum, Bromus, Coix, Saccharum, Zea, Sorghum, Setaria, Panicum, and Echinochloa. Among the plants belonging to the family Poaceae, preferred are plants belonging to the genus Oryza, more preferably rice (Oryza sativa L.). From the viewpoint that the plant to which the present invention is applied is a plant of which yield is affected by changes in the methylation level of Hd1, rice, wheat, rye, wild oats, barley, soybean, maize, sorghum, pumpkin, and Arabidopsis are suitable.

Other examples of the plant to which the present invention can be preferably applied are those belonging to the class Dicotyledonae (dicotyledons), specifically those belonging to any of the families Asteraceae, Lamiaceae, Brassicaceae, Apiaceae, and Fabaceae.

More specific examples of the plant are as follows

Poaceae: rice, wheat, rye, wild oats, barley, and corn
Asteraceae: lettuce, sanchu, and chisha
Lamiaceae: basil
Brassicaceae: Brassica rapa var. perviridis, Brassica rapa subsp. japonica, Raphanus sativus var. sativus, Brassica napus, Brassica rapa (native oilseed rape), Chinese cabbage (Brassica rapa subsp. pekinensis), turnip (Brassica rapa subsp. rapa), yellow sarson (Brassica rapa var. yellow sarson), purple-stem mustard (Brassica chinensis f. honsaitai), Pak choi (Brassica rapa var. chinensis), Brassica rapa var. chinensis, Brassica napus (subspecies oleifera), and Brassica napus (subspecies campestris)
Apiaceae: pakuchi (Coriandrum sativum)
Amaranthaceae: spinach
Fabaceae: soybean, pea, chickpea, kidney bean, yahuzu pea, clover, faba bean (Vicia faba), alfalfa (Medicago sativa), cowpea (Vigna unguiculata), ryokuto (Vigna radiata), black gram (sprout bean, Vigna radiata), and moth bean (Vigna aconitifolia)

For the present invention, the term “plant” is used in the sense of an individual plant or a part thereof, unless especially indicated, and the term “part thereof” encompasses seed (including germinated seed and immature seed), organ or a part thereof (including leaf, root, stem, flower, stamen, pistil, and fragments thereof), cultured plant cells, callus, and protoplast, unless especially indicated. The plant may be a genetically engineered plant or transformed plant. The plant may be a harvested product or propagation material. For the present invention, the terms propagation material refers to all or a part of a plant body that is used for propagation (sometimes referred to as seedling), unless especially noted, and examples include, for example, seed, seedling, cell, callus, and plumule. For the present invention, the term “harvested product” is used in the usual sense, unless especially noted, and may be all or a part of a plant body that is not used for propagation. For example, if the plant is rice, the harvested product encompasses harvested rice and paddy. The harvested product may also be referred to as crop or agricultural crop. For the present invention, the term “processed product” refers to a processed product that is produced directly or indirectly from a harvested product, unless especially noted. For the present invention, the processed product encompasses processed products that reflect characteristics of the harvested product. For example, when the plant is rice, milled rice, cocked rice, rice flour, breads, confectioneries, buns, and pizza using rice flour, and liquor (sake) are included in the scope of the processed product.

For the present invention, the term “progeny” used for a plant means a plant of which at least one genetic parent and/or ancestor is the plant in question, unless especially noted. The progeny may be a progeny obtained from a transformed plant as the parent (T1 etc.) and a progeny thereof, as well as a crossed progeny of which one of the parent is T0 or T1 (e.g., F1) and a progeny thereof, as long as the desired trait is inherited.

Examples of the product of the seed include bagged multiple seeds, e.g., 100 or more seeds, 1,000 or more seeds, or 10,000 or more seeds. The product may be for the production of a crop. If the plant is rice, seeds for production as the product may be referred to as raw paddy or seed paddy.

In this case, the product of the seed may have quality higher than the standard. Examples of the standard are given below.

Moisture: 14.0 to16.0% in terms of paddy moisture.
Floating rice husk ratio: within 5% found in water selection (5 out of 100 grains).
Sieve retention: 2.2 mm, 98% or more on sieve.
Germination: germination rate is 90% or higher.
Others: Free of dehulled grains or broken grains, foreign grains, and foreign matter
Mixing of non-glutinous rice: Contamination limit is 0.04% (4 out of 10,000 grains)

### [Useful Characteristic]

A plant having a useful characteristic can be grown from the environmental memory seed provided by the present invention. For the present invention, examples of such a useful characteristic include increased biomass, accelerated flowering, increased seed yield, and acquired heat tolerance during grain filling.

If the plant is a Poaceae plant, the useful characteristic of the plant obtained from the environmental memory seed is any selected from the group consisting of increased tiller number, increased yield per unit cultivation area, earlier heading stage, and low rate of chalky grains (white immature grains). The rate of chalky grains (%) can be calculated for threshed grains classified into five groups according to the classification mentioned in the section of Examples of this description in accordance with the equation: (Opaque number)/(Total number)×100.

In a preferred embodiment, the useful characteristic of a plant obtained from the environmental memory seed of wheat is increased yield. Specifically, yield obtainable with the environmental memory wheat seed obtained from wheat that was subjected to temperature stress (e.g., 15° C.) during the grain filling stage can increase by 50% or more, preferably 100% or more, more preferably 200% or more, further preferably 300% or more, such as about 340%, compared with yields of those cultivated from normal seeds under the same conditions. The same effect can also be expected for Poaceae plants other than wheat, or plants other than Poaceae plants.

In a preferred embodiment, the useful characteristic of a plant obtained from the environmental memory seed of barley is increased yield. Specifically, yield obtainable with the environmental memory barley seed obtained from barley that was subjected to temperature stress (e.g., 25° C.) during the grain filling stage can increase by 20% or more, preferably 30% or more, more preferably 40% or more, further preferably 45% or more, such as about 49%, compared with yields of those cultivated from normal seeds under the same conditions. The same effect can also be expected for Poaceae plants other than barley, or plants other than Poaceae plants.

In a preferred embodiment, one of the useful characteristics of a plant obtained from the environmental memory seed of rice is increased biomass under low nitrogen conditions. The degree of increase in biomass can be determined according to any selected from the group consisting of aboveground length, underground length, and dry weight. Specifically, dry weight on day 21 after germination of a plant obtained from an environmental memory rice seed can increase by 30% or more, preferably 40% or more, more preferably 50% or more, further preferably 60% or more, such as about 70%, compared with dry weights of those cultivated from normal seeds under the same low nitrogen conditions. The same effect can also be expected for Poaceae plants other than rice, or plants other than Poaceae plants.

In a preferred embodiment, one of the useful characteristics of a plant obtained from the environmental memory seed of lettuce is increased biomass. Specifically, aboveground weight of a plant obtained from the environmental memory lettuce seed can increase by 30% or more, preferably 40% or more, more preferably 50% or more, further preferably 60% or more, such as about 70%, compared with aboveground weights of those cultivated from normal seeds for the same days. The same effect can also be expected for Asteraceae plants other than lettuce, or plants other than Asteraceae plants.

In a preferred embodiment, one of the useful characteristics of a plant obtained from such an environmental memory seed of lettuce is faster increase in biomass. Specifically, the number of days required to obtain a desired amount of biomass with a plant obtained from the environmental memory lettuce seed is reduced by 5 days or more, preferably 10 days or more, more preferably 20 days or more, further preferably 30 days or more, such as about 35 days, compared with the numbers of days required for those cultivated from normal seeds. The same effect can also be expected for Asteraceae plants other than lettuce, or plants other than Asteraceae plants.

In a preferred embodiment, one of the useful characteristics of a plant obtained from such an environmental memory seed of cowpea is larger number of seeds. Specifically, the number of seeds of a plant obtained from the environmental memory cowpea seed increases by 3% or more, preferably 5% or more, more preferably 7% or more, further preferably 10% or more, e.g., about 15%, compared with the numbers obtained with those cultivated from normal seeds under the same conditions. The same effect can also be expected for Fabaceae plants other than cowpea, or plants other than Fabaceae plants.

### Method for Producing Seed Under Environmental Stress

The present invention also relates to a method for producing a seed of a plant having a useful characteristic. The production method of the present invention comprises the following steps:

the step of cultivating a first generation plant under an environmental stress; and
the step of obtaining a seed with environmental stress memory from the cultivated plant or a progeny thereof

### [Environmental Stress]

Examples of the environmental stress include temperature stress (high or low temperature stress), low nitrogen stress, salinity stress, low light stress, intense light stress, drought stress, excess humidity stress, heat stress, cold stress, nutrient stress, heavy metal stress, disease stress, oxygen deficiency stress, ozone stress, CO₂ stress, and high wind stress. The environmental stress is preferably temperature stress (high or low temperature stress) or low nitrogen stress during the seed developing period. The seed developing period is sometimes referred to as grain filling stage.

If the plant is a Poaceae plant, one of the preferred examples of the environmental stress is heat stress during the seed developing period, where the heat stress is that the plant is cultivated at an environmental temperature higher by at least 5° C. than the average temperature of that period for 6 weeks or longer after heading.

In the case of wheat, one of the preferred examples of the environmental stress is temperature stress (e.g., 10 to 20° C., such as 15° C.) during the seed developing period. In the case of barley, one of the preferred examples of the environmental stress is heat stress (e.g., 20 to 30° C., such as 25° C.) during the seed developing period. In the case of lettuce, one of the preferred examples of the environmental stress is heat stress (e.g., 20 to 32° C., such as 25° C. or 30° C.) during the seed developing period. In the case of cowpea, one of the preferred examples of the environmental stress is heat stress (e.g., 20 to 30° C., such as 25° C.) during the seed developing period. In the case of soybean, one of the preferred examples of the environmental stress is temperature stress (e.g., 15 to 25° C., such as 20° C.) during the seed developing period.

For the present invention, the memory of environmental stress is preferably a change in the methylation level of DNA in the promoter of the Heading date 1 (Hd1) gene. The change is an increase or decrease.

### EXAMPLES

### Rice

### [Materials and Methods]

### 1. Plant Materials, Growing Conditions and Growth Measurements

Rice plants (Oryza sativa L. cv. Nipponbare) were cultivated under natural conditions. The cultivation sites were Kyushu University Hakozaki Campus (Fukuoka, Japan, 33°67′ N, 130°42′ E) during 2015 to 2018 and Kyushu University Ito Campus (Fukuoka, Japan, 33°37′ N, 130°25′ E) during 2019 to 2020. Rice plants were grown under control conditions and subjected to a heat stress treatment during the grain filling stage, as previously reported (Suriyasak et al. 2020). For growth measurements, one 3-week old control rice seedling and one 3-week old rice seedling subjected to the heat stress treatment were transplanted into 1/5000 Wagener's pots with base fertilizer. The base fertilizer was as described in the previous study (Suriyasak et al. 2020). After the transplantation, the number of tillers and plant height were measured every 5 days, and the plants were harvested for yield analysis at 49 days after flowering (DAF). Ten 3-week old seedlings were transplanted into one pot for observation of leaf and heading phenotypes. The fully developed tillers were removed at the maximum tillering stage, and only the main stem was allowed to continue to grow thereafter for leaf sample collection and heading date confirmation. To evaluate the phenotype of the flag leaf, the length of the fully developed flag leaf was measured after heading. The center of the flag leaf was collected and stored in FAA (formalin-acetic acid-ethyl alcohol) solution until analysis. Then, 15-um thick sections were prepared from the leaf samples by a frozen sectioning method using OCT compound (Leica CM1950, Leica Microsystems), and visualized under a microscope (BZ-X710, Keyence), and leaf thickness, width, and cell layers were measured by using ImageJ.

### 2. Yield Analysis

For cultivation in pots, a single 3-week-old seedling from each treatment group was transplanted into 1/5000a Wagener's pot with the same fertilizer application as described above. For field experiments, 3-week-old seedlings from the respective treatment groups were transplanted into experimental paddy fields at the university. In this experiment, a distinct experimental plot was prepared for each treatment, and 49 rice plants per experimental plot were planted at a density of 30×30 cm interval. Prior to the transplantation, a slow released sigmoid type fertilizer was mixed. From the center of the experimental plot, 15 contiguously planted rice plants were harvested for yield analysis, and the number of panicles and total number of spikelets per plot were counted. After threshing, brown rice was collected using a 1.8-mm sieve to determine filling rate and other yield components. Grain moisture content was calculated from the dry weight of the grains dried at 80° C. for 24 hours, and total yield and one-thousand grain weight were calculated by using a moisture content of 15%.

### 3. Comprehensive DNA Methylation Analysis by Whole Genome Bisulfite Sequencing (WGBS) Method

Genomic DNA from each sample was extracted from 20 dried seeds using the ISOSPIN plant DNA extraction kit (Nippon Gene). Pooled gDNA extracted from 10 samples derived from each treatment was subjected to a bisulfite treatment prior to the sequence analysis. Reference genome sequence, genes and TE (transposable element) annotations were obtained from RAP-DB. Those with a methylation level that differed by 1.25-fold or more (difference in methylation level between the control and treatment plot did not fall within the range of ±25%, P value was less than 0.05) were extracted as differentially methylated positions (DMPs) and differentially methylated regions (DMRs), and the extracted DMRs were screened for 100 bp at a time using the sliding window method. DMRs located within 3000 bp upstream and downstream of the translation region, respectively, were considered as transcription start sites (TSS) of the promoter. Gene ontology (GO) analysis was performed by using the “GO biological process complete”, “GO molecular function complete” and “GO cellular component complete” analysis set of Panther Software and the setting of ‘Fisher's Exact’ test with ‘No correlation’. The genes involved in each GO are listed in the table in descending order of number.

### 4. RNA Sequencing and Quantitative Real-Time PCR

Analysis tailored for each growth stage was performed. Roots and leaves at the maximum tillering stage (OsSLB1) or the newest, fully developed leaves were collected at 10 AM (OsHd3a) and 8 PM (OsHd1) according to the circadian expression of the respective genes. In addition, growing roots were collected at 55 days after sowing (DAS) during the maximum tillering stage, and seeds during grain filling were collected at 15 days after flowering (DAF). Total RNA was extracted from the cryopreserved leaves, roots, or seeds by during grain filling by the SDS/phenol/lithium chloride method (Chirgwin et al. 1979), and RNA sequencing was performed by using two biological replicates per treatment (55 DAS, PM). All the differentially expressed genes (DEGs, P <0.05) were analyzed, and a heatmap of RNA reads was generated by using OriginPro. GO analysis for DEGs was performed by using the same method as described above for DMR. For quantitative real-time PCR (qRT-PCR), cDNA was synthesized from the extracted RNA by using the ReverTra Ace reverse transcriptase (Toyobo) according to the manufacturer's instructions. The quantitative real-time PCR was performed by using the CFX Connect Optics Module real-time PCR detection system (Bio-Rad) with SYBR Green (Toyobo) according to the manufacturer's instructions. The primers used for qRT-PCR are listed in Supplemental Table 2-1. The PCR conditions were as follows: initial denaturation at 94° C. for 2 minutes, followed by 40 cycles of denaturation at 94° C. for 20 seconds, annealing at the temperature set for each primer for 20 seconds, and extension at 72° C. for 20 seconds, followed by melting and plate reading. The results were normalized based on the expression level of OsUBQ (leaf samples) or OsActin (root and developing grain samples).

### 5. DNA Methylation Analysis by MeDIP-qPCR

Genomic DNA was extracted from dry seeds, growing leaves, and developing grains using the DNeasy Plant Mini Kit (Qiagen) and sheared into approximately 500 bp fragments by sonication. For MeDIP-qPCR, immunoprecipitation was performed with 1000 ng of the sheared DNA using an immunoprecipitation kit (Active Motif) as described in the manufacturer's protocol. The immunoprecipitated DNA was subjected to qRT-PCR to detect locations of changes in DNA methylation level. Percent input was calculated as described in the manufacturer's protocol (https://www.diagenode.com/files/products/kits/magmedip-qpcr-kit-manual.pdf). Specifically, % input is a ratio of DNA precipitated with the methylation antibody directed to the DNA used in the analysis (input). The specific primers used for MeDIP-qPCR are listed in the following table.

TABLE 2

| | | SEQ ID | | SEQ ID |
|---|---|---|---|---|
| | Forward 5′ | NO: | Reverse R′ | NO: |
| qRT-PCR primers | | | | |
| OsActin | GACTCTGGTGATGGTGTCAGC | 46 | GGCTGGAAGAGGACCTCAGG | 47 |
| OsUBQ | AACCAGCTGACGCCCAAGA | 48 | ACGATTCATTTAACCAGTCCATGA | 49 |
| OsSLB1 | CAGGTTCTCAAGAGGCTTCG | 50 | GGAGGTGCTCGTAAGTGAGC | 51 |
| OsHd1 | TCAGCAACAGCATATCTTTCTCATCA | 52 | TCTGGAATTTGGCATATCTATCACC | 53 |
| OsHd3a | GCTCACTATCATCATCCAGCATG | 54 | CCTTGCTCAGCTATTTAATTGCATAA | 55 |
| OsAGPS2b | AACAATCGAAGCGCGAGAAA | 56 | GCCTGTAGTTGGCACCCAGA | 57 |
| OsGBSSI | AACGTGGCTGCTCCTTGAA | 58 | TTGGCAATAAGCCACACACA | 59 |
| OsSuSy2 | TTCAGCAGGAGAAGCCGTCAGC | 60 | CCGGCGTTTATTTGAGGCAAGC | 61 |
| OsAmy1A | GATACGACGTCGAACACCTC | 62 | CGGATCGGATACAGCTCGTTG | 63 |
| OsAmy3D | GTAGGCAGGCTCTCTAGCCT | 64 | CCAACGGTTACAAACTGCGTGA | 65 |
| MeDIP-qPCR primers | | | | |
| OsSLB1 promoter P1 | TCATTCACAACACACTTGAAGCTTGA | 66 | TCCGACTTCCTCTACTCCACCTCT | 67 |
| OsSLB1 promoter P2 | ACGGTGTAGACATGGTGTTTGGGT | 68 | ATGGCTCTACAGCGTAACCGGGTA | 69 |
| OsSLB1 promoter P3 | TACCCGGTTACGCTGTAGAGCCAT | 70 | TGTCAGCACGGAAGATACCGATGA | 71 |
| OsHd1 promoter P1 | CAGATCTCCAAGTTGCGCCATAAC | 72 | CGTCGCCGATTTCAATTCGAAAAT | 73 |
| OsHd1 promoter P2 | GTACATCATCACAGTGGCTTCCAATTCT | 74 | AAGTATCTAACCTGAACTGATGTCAATACC | 75 |
| OsHd1 promoter P3 | TGTCAATCGCTGGATTCGACTTGA | 76 | GAACGAAGTAGCCTTGCTTGTGGT | 77 |
| OsHd3a promoter P1 | CTTGTATACATGTGTCTTCTTAGCG | 78 | CCTCCAATCTGTAGGAATTCGAT | 79 |
| OsHd3a promoter P2 | ACGCTTAGCTATAGCTGCTGCTGC | 80 | TTAGCTCGGTCAGTGTACTGCATG | 81 |
| OsAGPS2b promoter | GTGTAACGTTTGCCCACAACAACC | 82 | TGCCAATACATTCATGGTTGCAACA | 83 |
| OsGBSSI promoter | AACTAGCTCCACAAGATGGTGGGC | 84 | AGTGGCGAGAGACATGATTTAACGA | 85 |
| OsSuSy2 promoter | GCTGACAATGGTGCCGGCATATGC | 86 | TCATGTCACTAGCACAGGCGAGTAC | 87 |
| OsAmy1A promoter | GGCTCTTGCCAGAAACAACCTGTG | 88 | TAGAGCAATAAAGGATCGGTCGGC | 89 |
| OsAmy3D promoter | CATTTCGAGGTCCGGATTAGGGTC | 90 | GGTTGCAGTTAGCCTCTTGACTGG | 91 |

### 6. Evaluation of Heat Tolerance During Grain Filling Stage

After allowing growing and flowering in a natural environment, control and high temperature treated plants were divided into three groups, control-control (25° C.), control-heat (30° C.), and heat-heat (30° C.), where the former and the latter refer to parent and offspring's temperature regimes during grain filling, respectively, and used for heat tolerance experiments. The SPAD measurement was performed every 5 days. Three flag leaves were selected from each pot and the measurement was performed for three locations, the top, middle, and bottom, of the leaf to generate an average SPAD value, and for the same locations on the same flag leaf each time thereafter. On day 26 after flowering (DAF), the photosynthetic rate of the central part of the flag leaf (3 or 4 replicates) was measured. This measurement was performed by using an LCi portable photosynthesis system (Li-Cor Biosciences, Lincoln, Nebraska, USA) between 8:00 to 14:00 on a sunny day. The measurement conditions were set at a chamber temperature of 25° C., and a photosynthetically active radiation (PAR) of 1,500 photon flux density (μmol m-2s-1), and the reference background was CO₂ of the natural atmosphere. Gene expression in developing grains was analyzed for each treatment as described above, and classification of chalky grains was performed as described in the previous study. Specifically, grains were visually observed from below under transillumination and evaluated based on the fact that normal rice grains transmit light and thus are translucent, while immature ones have opaque or chalky areas that prevent transmission of light, creating dark areas. According to the criteria of Ishimaru et al. (2009), the grains were classified into five groups: opaque, white-belly, white-based, white-backed, and immature.

### [Results]

### 1. Heat Stress During Grain Filling Alters Plant Phenotype and Increases Yield of Progeny

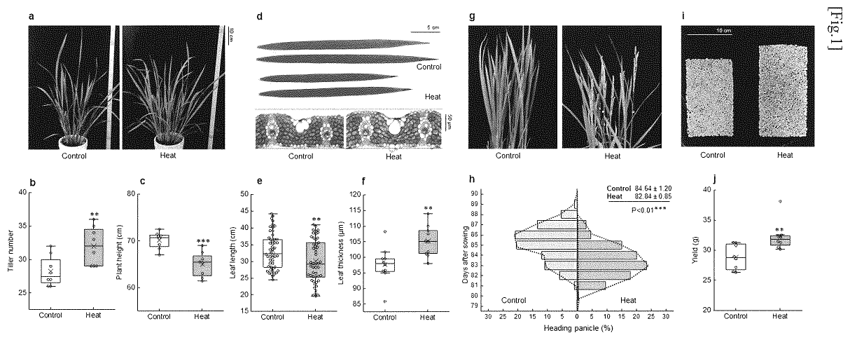

To elucidate how heat stress during grain filling affects phenotypic changes of the progeny, control and heat stressed seeds were sown and grown under natural conditions. As a result, it was observed that at the vegetative stage, plants from seeds heat stressed during grain filling developed more tillers during growth thereafter, and at the maximum tillering stage, the plant height was reduced compared with the control (FIG. 1, a to c). Further, as for the phenotypes of leaves, the plants from seeds heat stressed during grain filling showed significantly shorter and thicker flag leaf phenotype with increased cell layers adjacent to vascular bundle, compared with the control plants, despite no change in leaf width (FIG. 1, d to f). At the heading stage, the plants from seeds heat stressed during grain filling developed early heading phenotype, which was nearly about two days earlier than the control plants grown under the same natural condition (FIG. 1, g and h). At the harvesting stage, the plants from seeds heat stressed during grain filling showed significant increase in total yield per single plant per pot by 14.47% based on that of the control (FIG. 1, i and j). Similar results were also obtained in the field experiment, and the plants from seeds heat stressed during grain filling showed increase in yield per square meter by 9.51% compared with the control (Table. 2). Both the pot experiment and field experiment showed that panicle numbers were significantly increased in the plants grown from the seeds heat stressed during grain filling, while spikelet number per panicle, grain filling rate and one-thousand grain weight remained unchanged (Table 2), suggesting that increase in panicle number mainly contributed to higher yield in the progeny plants from seeds heat stressed during grain filling. Thus, heat stress during grain filling significantly altered progeny phenotypes by increasing tiller number, altering flag leaf phenotype, enhancing early heading, increasing final yield, etc. compared with the control, and these changes were also observed in reproduced experiments conducted in other years.

TABLE 3
Yield components of plants grown at natural condition

| | Panicle | Spikelet/ | Grain filling | 1000-grain | Total yield |
|---|---|---|---|---|---|
| | number | panicle | (%) | weight (g) | (g) |
| Pot | | | | | |
| experiment | | | | | |
| Control | 19.25 ± 148 | 67.76 ± 4.42 | 88.68 ± 3.67 | 25.05 ± 1.41 | 28.25 ± 2.11 |
| Heat | 21.23 ± 2.13 | 67.07 ± 5.32 | 88.73 ± 2.71 | 25.25 ± 1.33 | 32.34 ± 2.49 |
| Student's t-test | P = 0.012* | P = 0.39 | P = 0.46 | P = 0.38 | P = 0.005** |
| (n = 8) | | | | | |
| Field | | | | | |
| experiment | | | | | |
| Control | 331 ± 9.53 | 92.27 ± 3.42 | 93.22 ± 2.01 | 19.66 ± 0.03 | 570.54 ± 19.58 |
| Heat | 356 ± 12.16 | 92.51 ± 7.64 | 93.41 ± 0.55 | 19.82 ± 0.12 | 624.84 ± 32.27 |
| Student's t-test | P = 0.025* | P = 0.40 | P = 0.44 | P = 0.06 | P = 0.040* |
| (n = 3) | | | | | |

### 2. Heat Stress During Grain Filling Induces Changes in Global Methylomes in Dry Seeds

The inventors of the present invention hypothesized that epigenetic regulation, especially DNA methylation, might play a crucial role in this transgenerational memory, and therefore methylated cytosines were compared at single base resolution to generate methylomes in both the control and heat stressed seeds. Global methylation analysis via whole genome bisulfite sequencing (WGBS) showed that in the control seeds, 56.6%, 27.0% and 14.1% of cytosine residues were methylated in the CG, CHG and CHH contexts, respectively, while in the heat stressed seeds, total 3,991,072,994 cytosines were sequenced and 57.8%, 28.5% and 15.5% of cytosine residues were methylated in the CG, CHG and CHH contexts, respectively (Table 4).

TABLE 4
Total cytosines analyzed by whole genome bisulfite sequencing

| | Control | Heat |
|---|---|---|
| Total cytosines analysed | 3,919,488,890 | 3,991,072,994 |
| Duplication rate (%) | 12.1% | 10.8% |
| Coverage cytosine for mapping (%) | 97.9% | 97.9% |
| Methylated cytosines in CpG context | 356,642,852 | 369,500,795 |
| Methylated cytosines in CHG context | 166,128,581 | 177,195,999 |
| Methylated cytosines in CHH context | 375,821,081 | 423,437,019 |
| Unmethylated cytosines in CpG context | 273,533,402 | 270,050,603 |
| Unmethylated cytosines in CHG context | 449,808,489 | 443,743,999 |
| Unmethylated cytosines in CHH context | 2,297,554,485 | 2,307,144,579 |
| CpG methylation (%) | 56.6% | 57.8% |
| CHG methylation (%) | 27.0% | 28.5% |
| CHH methylation (%) | 14.1% | 15.5% |

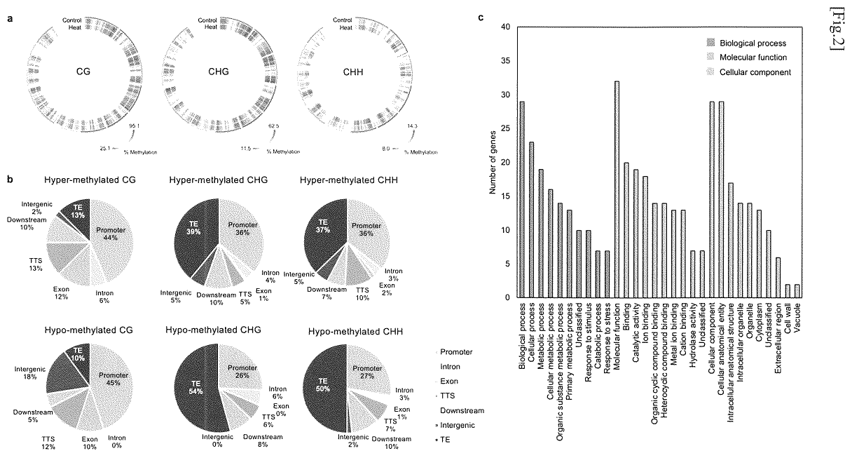

Overall, heat stress during grain filling caused hypermethylation of about one percent for all methylated cytosine contexts. Average methylation levels of cytosines in 1 Mbp window size of all contexts along chromosomes were plotted as chromosome maps, showing genome-wide altered methylation levels in heat stressed seeds compared with the control (FIG. 2, a). Then, differentially methylated regions (DMRs) of the seeds heat stressed during grain filling were identified by comparison with the control, and 268 hypermethylated and 189 hypomethylated DMRs, total 457 DMRs, were identified. Distributions of DMRs showed that DMRs of CG context were mostly overlapped with gene promoter, followed by a high proportion of both high and low methylation in the TE region (FIG. 2, b).

On the other hand, in non-CG contexts, most of DMRs were found in TE regions, followed by promoter regions. In addition, DMRs overlapped with TEs were found to be the most abundant in retrotransposons. GO analysis of DMRs that overlapped with protein coding genes revealed that DMRs relating to stress response, metal ion transport, response to oxidative stress, and other various biological processes are abundantly present (FIG. 2, c).

The above results suggested that heat stress during grain filling induced global methylome alterations especially in promoter regions of coding genes.

### 3. Transcriptional Changes During Vegetative Growth are Caused by Heat Stress During Grain Filling

To obtain better understanding about how changes in global DNA methylation would affect transcriptional regulations during plant development, RNA-sequencing analysis was performed for leaf RNA obtained during vegetative growth.

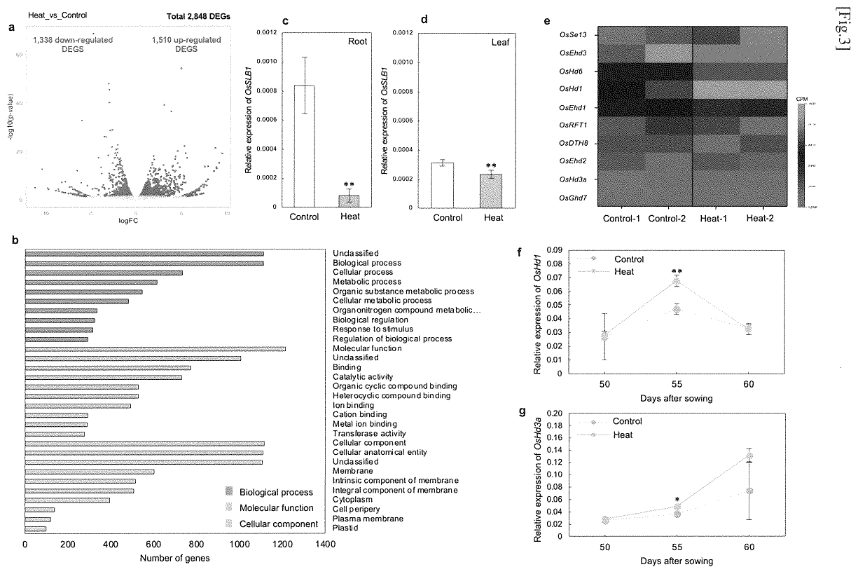

As a result, 1,510 up-regulated and 1,338 down-regulated differentially expressed genes (DEGs) were found, and thus total 2,848 DEGs were identified (FIG. 3, a). Gene ontology analysis of the total DEGs showed that many genes involved in various regulatory pathways such as oxidative stress response, photosynthesis, metabolic process and intracellular anatomical structure were included (FIG. 3, b).

Among the down-regulated DEGs, strigolactone biosynthesis 1 (OsSLB1) is an ortholog of MAX1 involved in aboveground shoot lateral branching of Arabidopsis, suggesting that it may be involved in tillering suppression in rice. The previous studies suggested that strigolactone (SL) is mainly synthesized in roots and moves acropetally to aboveground part (Wang and Li et al. 2011) to suppress tillering while induce height in rice (Liu et al. 2020). Therefore, OsSLB1 gene expression in roots was analyzed, and it was found that its expression was significantly suppressed in the plants from seeds heat stressed during grain filling for about 10-fold (FIG. 3, c). In addition, OsSLB1 expression was also significantly suppressed in leaves of the plants heat stressed during grain filling (FIG. 3, d).

Among the up-regulated DEGs, a heading-related gene ‘Heading date 1’ (OsHd1), an ortholog of Arabidopsis's CONSTAN, which plays important role in heading date regulation in rice, was found. To elucidate the candidate gene playing a role in early heading phenotype of the plants heat stressed during grain filling, expression heatmap of genes involved in heading regulations in rice was generated (FIG. 3, e), showing that expression of ‘Heading date 1’ (OsHd1), an ortholog of Arabidopsis's CONSTAN, was up-regulated in the plants heat stressed during grain filling, and it was also found in significantly up-regulated DEGs. Therefore, expression levels of OsHdl and its downstream ‘Heading date 3a’ (OsHd3a), an ortholog of Arabidopsis's FT, prior to heading were confirmed. As a result, it was shown that expressions of both OsHd1 and OsHd3a were up-regulated in the plants heat stressed during grain filling (FIG. 3, f and g) to induce early heading in the plants heat stressed during grain filling.

These results indicate that down-regulation of OsSLB1 and up-regulation of OsHd1 might play important roles for increased tillering and early heading phenotypes in progeny plants heat stressed during grain filling.

### 4. Alterations of DNA Methylation in Dry Seeds Remain in Developing Organs for Transcriptional Regulations

The results mentioned above showed that transcriptional changes of OsSLB1 and OsHd1 during plant development were involved in promotion of tillering and heading, respectively, in heat stressed plants, and it was expected that altered DNA methylation levels of these gene promoters might play important role in transcriptional regulations. Therefore, DNA methylation levels of OsSLB1, OsHd1 and OsHd3a gene promoters in both dry seeds and in developing organs were analyzed by performing MeDIP-qPCR (Methylated DNA Immuno-Precipitation-qPCR) with loci-specific amplicons.

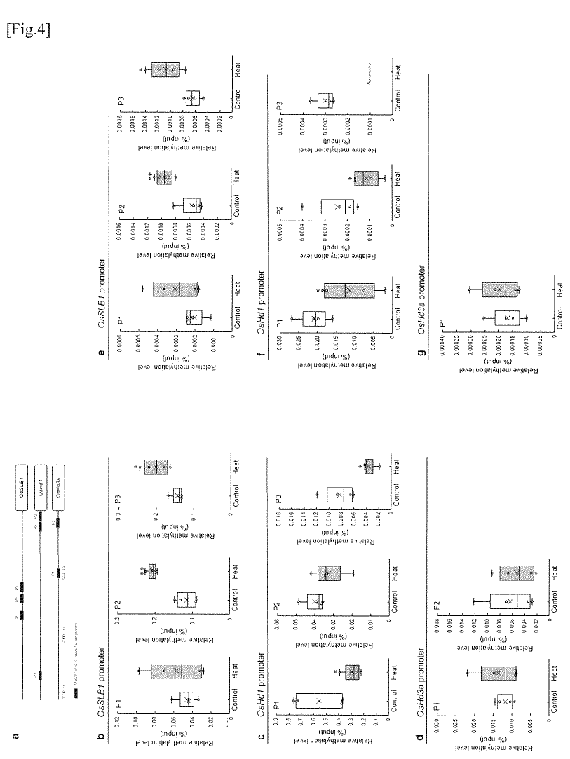

In the heat-stressed dry seeds, it was shown that the OsSLB1 promoter regions were significantly hypermethylated, while OsHd1 promoter regions were significantly hypomethylated (FIG. 4, b and c). In developing roots of the plants from seeds heat stressed during grain filling at the maximum tillering stage, significant hypermethylation in the OsSLB1 promoter regions was observed (FIG. 4, e), together with significant hypomethylation in the OsHd1 promoter regions in leaves (FIG. 4, f). Yet, methylation levels of the OsSLB1 promoters showed no difference in leaves between the control and plants from seeds heat stressed. To be noted, there was no significant changes in methylation levels of OsHd3a promoter itself, in both dry seeds and leaves (FIG. 4, d and g), suggesting that the transcriptional up-regulation of OsHd3a might not be due to its DNA methylation status itself, but due to methylation status of upstream OsHd1.

In summary, these data suggested that heat stress during grain filling induces hypermethylation of OsSLB1 promoter and hypomethylation of OsHd1 promoter to provide increased tillering and early heading phenotype, which DNA methylation status remained at some contents from dry seeds to developing organs.

### 5. Progeny Developed From Heat Stressed Plants Shows Thermotolerance During Grain Filling Stage

Since it was found that many DMRs and DEGs were involved in abiotic stress responses and detoxification, the inventors of the present invention expected that heat stress during grain filling of parent plants might induce transgenerational stress memory for progeny to cope with repeated such stress. To observe whether progeny of heat stressed plants acquires thermotolerance during grain filling stage or not, after anthesis, plants were divided into three groups, control-control (25° C.), control-heat (30° C.), and heat-heat (30° C.), where the temperatures for the former and the latter refer to development temperature of the parent and progeny plants during grain filling, respectively.

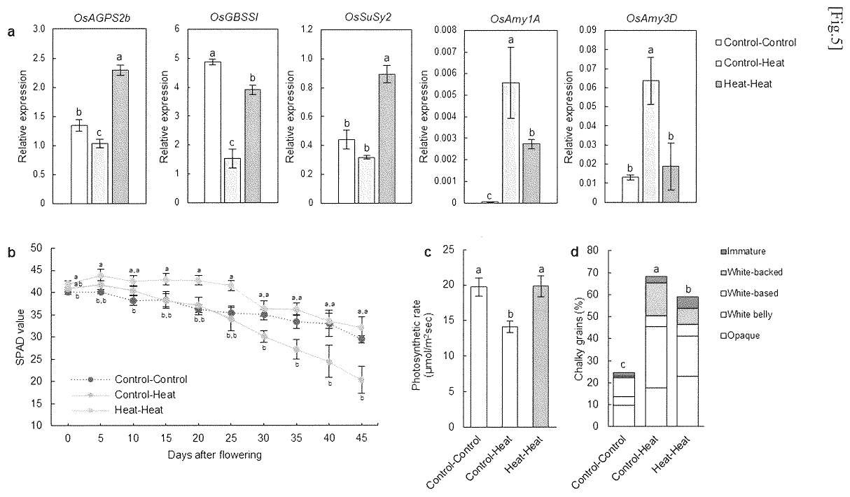

As a result, it was observed that heat stress during grain filling accelerated flag leaf senescence with dropped SPAD value, together with decrease in photosynthetic rate in the control-heat plants, compared with the control-control plants (FIG. 5, a and b). However, heat-heat plants showed significantly delayed leaf senescence and higher photosynthetic rate compared with the control-heat plants, even under the same heat stress condition (FIG. 5, a and b). This suggested that leaf senescence and decrease in photosynthetic rate due to heat stress were suppressed in the heat-heat plants.

It is known that heat stress during grain filling causes chalky grains to decrease grain quality in rice due to down-regulation of starch biosynthesis genes and up- regulation of starch degrading genes (Hakata et al. 2012, Tanamachi et al. 2016, Suriyasak et al. 2017). In the present study, after harvest, the heat-heat plants developed significantly reduced chalky grains compared with the control-heat plants (FIG. 5, c and d), with no change in total grain yield. Therefore, expression levels of starch biosynthesis genes, OsAGPS2b (ADP-glucose pyrophosphorylase subunit 2b), OsGBSSI (granule-bound starch synthase) and OsSuSy2 (sucrose synthase 2), and starch degrading genes, OsAmy1A (α-amylase 1A) and OsAmy3D (α-amylase 3D) in developing seeds at 15 DAF were determined. As a result, the control-heat plants showed significantly down-regulated starch biosynthesis gene expression and significantly up-regulated starch degrading gene expression, compared with those of the control (FIG. 5, a). Yet, the heat-heat plants did not show suppression of starch biosynthesis gene expression and induction of starch degrading gene expression due to heat stress, resulting in significantly higher starch biosynthesis and suppressed starch degrading gene expressions compared with the control-heat plants (FIG. 5, a).

These results suggest that delayed leaf senescence together with transcriptional changes of starch metabolism in developing grains of heat-heat plants contributed to their thermotolerance for better grain quality under heat stress, and similar phenomena were also observed in reproduced experiments conducted in other years.

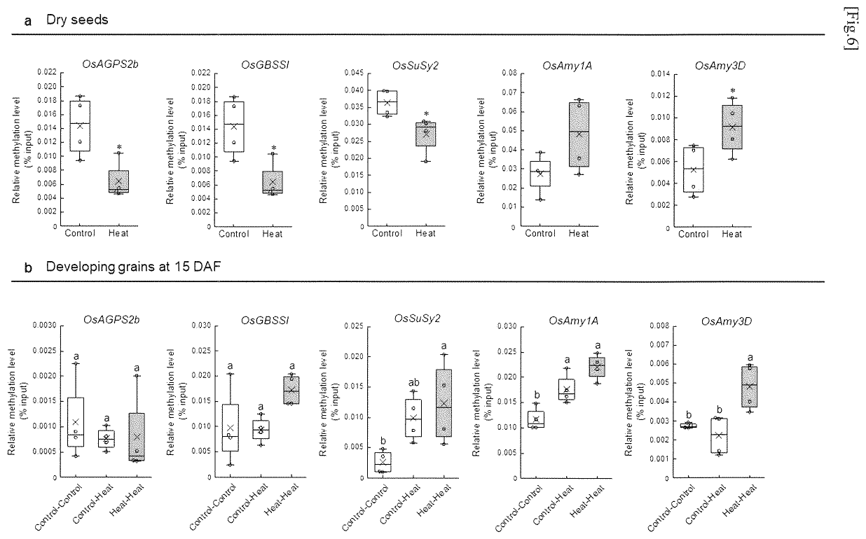

To explain how progeny of heat stressed plants shows altered transcriptional levels under repeated heat stress, MeDIP-qPCR was performed for promoter regions of starch metabolism genes in dry seeds and in developing grains. In the heat stressed seeds, promoters of starch biosynthesis genes were significantly hypomethylated, while starch degrading gene promoters were hypermethylated (FIG. 6, a).

In the developing grains, despite no change in methylation levels of starch biosynthesis genes between the control-heat and heat-heat plants, promoter region of the starch degrading gene, OsAmy3D, remained significantly hypermethylated in the heat- heat plants (FIG. 6, b).

In summary, these results suggested heat stress during grain filling alter DNA methylation in seeds to induce thermotolerance to maintain grain quality under repeated heat stress condition.

The following table shows the numerical data of DNA methylation analysis conducted by MeDIP-qPCR for methylation levels of promoter regions.

TABLE 5
Relative methylation level (% input) by MeDIP-qPCR
1. Harvested seeds: Four replicates (40 seeds/replicate)

| Specific | Control | | | Heat | | |
|---|---|---|---|---|---|---|
| loci | Maximum | Minimum | Average | Maximum | Minimum | Average |
| OsSLB1:proP1 | 0.0623 | 0.0336 | 0.0459 | 0.0973 | 0.0282 | 0.0494 |
| OsSLB1:proP2 | 0.1484 | 0.0891 | 0.1159 | 0.2266 | 0.1949 | 0.1981 |
| OsSLB1:proP3 | 0.1686 | 0.1327 | 0.1438 | 0.2437 | 0.1632 | 0.1709 |
| OsHd1:proP1 | 0.7613 | 0.3760 | 0.5584 | 0.3930 | 0.2224 | 0.3077 |
| OsHd1:proP2 | 0.0486 | 0.0357 | 0.0400 | 0.0421 | 0.0192 | 0.0323 |
| OsHd1proP3 | 0.0119 | 0.0060 | 0.0083 | 0.0042 | 0.0018 | 0.0035 |
| OsHd3a:proP1 | 0.0145 | 0.0091 | 0.0118 | 0.0219 | 0.0088 | 0.0108 |
| OsHd3a:proP2 | 0.0121 | 0.0028 | 0.0063 | 0.0092 | 0.0021 | 0.0047 |
| OsAGPS2b:pro | 0.0216 | 0.0113 | 0.0176 | 0.0133 | 0.0034 | 0.0096 |
| OsGBSSI:pro | 0.0399 | 0.0323 | 0.0364 | 0.0309 | 0.0190 | 0.0270 |
| OsSusy2:pro | 0.0186 | 0.0094 | 0.0143 | 0.0104 | 0.0046 | 0.0050 |
| OsAmy1A:pro | 0.0386 | 0.0139 | 0.0274 | 0.0664 | 0.0271 | 0.0648 |
| OsAmy3D:pro | 0.0074 | 0.0027 | 0.0052 | 0.0118 | 0.0062 | 0.0090 |

2. Developing organs: Four replicates

| Specific | Control | | | Heat | | |
|---|---|---|---|---|---|---|
| loci | Maximum | Minimum | Average | Maximum | Minimum | Average |
| OsSLB1:proP1 | 0.00024 | 0.00011 | 0.00020 | 0.00048 | 0.00019 | 0.00034 |
| OsSLB1:proP2 | 0.00083 | 0.00042 | 0.00056 | 0.00110 | 0.00079 | 0.00090 |
| OsSLB1:proP3 | 0.00078 | 0.00046 | 0.00064 | 0.00140 | 0.00075 | 0.00106 |
| OsHd1:proP1 | 0.02684 | 0.01597 | 0.02092 | 0.01870 | 0.00206 | 0.01152 |
| OsHd1:proP2 | 0.00040 | 0.00015 | 0.00024 | 0.00016 | 0.00003 | 0.00011 |
| OsHd1proP3 | 0.00036 | 0.00026 | 0.00030 | No detection | | |
| OsHd3a:proP1 | 0.00025 | 0.00010 | 0.00016 | 0.00030 | 0.00012 | 0.00019 |

| | | |
|---|---|---|
| OsHd3a:proP2 | No detection | No detection |

3. Developing grains 15 DAF: Four replicates

| Specific | Control-Control | | | Control-Heat | | | Heat-Heat | | |
|---|---|---|---|---|---|---|---|---|---|
| loci | Maximum | Maximum | Minimum | Maximum | Minimum | Average | Maximum | Minimum | Average |
| OsAGPS2b:pro | 0.00225 | 0.00420 | 0.00109 | 0.00102 | 0.00050 | 0.00076 | 0.00201 | 0.00032 | 0.00079 |
| OsGBSSI:pro | 0.02041 | 0.00244 | 0.00975 | 0.01255 | 0.00631 | 0.01070 | 0.02043 | 0.01456 | 0.01726 |
| OsSusy2:pro | 0.00475 | 0.00097 | 0.00258 | 0.01430 | 0.00579 | 0.01005 | 0.02039 | 0.00556 | 0.01232 |
| OsAmy1A:pro | 0.01487 | 0.01008 | 0.01171 | 0.02183 | 0.01507 | 0.01625 | 0.02298 | 0.01887 | 0.02209 |
| OsAmy3D:pro | 0.00264 | 0.00292 | 0.00275 | 0.00317 | 0.00121 | 0.00250 | 0.00594 | 0.00347 | 0.0102 |

### [Discussion]

The results of the present study indicate that multiple phenotypic changes, such as accelerated tillering and shorter plant height, occur in rice progeny whose parent plants were subjected to heat stress during the grain filling stage compared with the control. The previous study has shown that the plant hormone strigolactone plays an important role in promoting rice tillering and suppressing plant height (Wang and Li, 2011). In the present, it is shown that strigolactone biosynthesis is inhibited by hypermethylation of its promoter region in plants subjected to heat stress, which may result in accelerated tillering and suppressed plant height during vegetative growth. Therefore, the reduction in plant height observed in plants treated with heat stress may have been due to homeostasis or energy balance between tillering and vertical growth.

Another study has described another function of OsHd3a in rice lateral branching. The Hd3a protein produced in leaf blades is transported from leaf phloem to apical meristem for axillary bud outgrowth (Tsuji et al. 2015), suggesting a possibility of up-regulation of heading related genes affecting tiller outgrowth in heat stressed plants. It is known that tillering is a crucial agronomic trait that determines grain production in Poaceae plants (Hussien et al. 2014). In the present study, higher panicle number due to accelerated tillering was the main factor to increase grain yield in the plants from seeds heat stressed during grain filling, while other yield components showed no difference between the control and heat-treated plants. Increase in panicle number resulted in higher spikelet number per plant, which might benefit reproductive strategy for plants and contribute to increase in production yield for human beings.

The heat stressed plants also showed altered flag leaf phenotype, with shorter but thicker leaves. These changes in leaf morphology might lead to delay of leaf senescence and increased photosynthetic rate under heat stress compared with the control-heat plants. In perennial ryegrass, significant negative correlation between leaf thickness and leaf H2O2 content, together with positive correlation between thickness and photosynthetic parameter, were observed under heat stress (Soliman et al. 2015). Also, amount of chlorophyll and photosynthetic rate during grain filling are important factors that affect rice grain yield and quality (Yang et al. 2015). Therefore, it was suggested that alteration of flag leaf structural properties induced by heat stress during grain filling play an important role to increase thermotolerance and improve grain quality of the next generation. Up to date, candidate genes regulating flag leaf length and thickness have not been identified yet, and therefore it is not sure which genes are involved in this phenomenon, but it might be possible that genes relating to the anatomical structure presented in the gene ontology analysis contribute to such changes in leaf morphology.

At the anthesis stage, heat stressed plants reached heading stage earlier than the control under natural conditions. Normally, high temperature before heading induces rapid heading in rice via up-regulated OsHd3a expression (Luan et al. 2009), and similar, but intergenerational phenomenon was observed in the present study. Another study has also shown that warm and cool climates of different growing areas influence progeny heading date in rice due to difference in water temperature during vegetative growth (Koumoto et al. 2018). In terms of epigenetic regulations on heading, although a role of histone modification, H3K27me3, on DTH8-Hd1 binding to the OsHd3a locus was proposed (Du et al. 2017), but the involvement of transgenerationally inheritable induced DNA methylation in heading phenotype in rice is still obscure even now.

The results of the present study showed that heat stress during grain filling significantly caused hypomethylation in the OsHd1 promoter in dry seeds and leaves, resulting in higher gene expression of OsHdl itself and its downstream OsHd3a prior to anthesis. OsHd3a gene expression was significantly induced in the heat stressed plants despite no change of methylation levels at its promoter regions compared with the control. Therefore, it is estimated that OsHd1 might play a key role for transgenerational induced DNA methylation to regulate its downstream OsHd3a, and thereby induce heading in rice. It has been found that Hd1 has dual effects of inducing and inhibiting heading under short-day (SD) and long-day (LD) conditions (Liu et al. 2014). Therefore, the inventors of the present invention also cultivated heat stressed plants under both SD and LD conditions, and observed that the higher expression of Hd1 in the heat stressed plants led to earlier heading phenotype under the SD condition, and late heading phenotype under the LD condition. These results suggested that OsHd1 might play an important role as a key factor for regulating heading date in rice in a day-length dependent manner via DNA methylation changes caused by heat stress during grain filling.

In Arabidopsis thaliana, progeny whose parents were subjected to heat stress also show early flowering phenotype due to FT up-regulation, suggesting presence of heat stress-induced transgenerational effects on flowering dates in dicots (Liu et al. 2019). Moreover, it has been found that this transgenerational effect is epigenetically regulated by H3K27me3 of HEAT SHOCK TRANSCRIPTION FACTOR A2 (HSFA2), and this effect lasts for at least two successive generations to act for removal of heat stress (Liu et al. 2019). Another study also reports the grandparental effects of heat stress on progeny flowering dates (Groot et al. 2017). In the present study, it was confirmed that two-generation (HCC) heat stress-removed plants showed early heading phenotype together with up-regulated OsHd1 and OsHd3a gene expressions to the levels similar to those of heat stressed plants. In addition, hypomethylation of the OsHd1 promoter was remained in HCC plants at the same content as that of heat stressed dry seeds. Therefore, it has been suggested that heat stress-induced transgenerational memory through DNA methylation on heading date also last long at least two successive generations, if the stress is removed. Yet, at present, it is still unclear for how many generations such stress removal is required for maintaining such transgenerational memory, and different phenotypes might cause difference in the number of generations for which stress removal is required. Therefore, further study is necessary.

It has been shown that progeny plants whose parents were exposed to heat stress acquired thermotolerance during the grain filling stage. Further, besides such flag leaf morphological changes as mentioned above, transcriptional changes of starch metabolism genes were also observed. Rice plants develop chalky grains, one of the main factors for quality reduction, under high temperature due to up-regulation of starch degradation gene expression and down-regulation of starch biosynthesis gene expression (Tanamachi et al. 2017, Yamakawa et al. 2007). In the present study, it was observed that hypomethylation and hypermethylation of starch biosynthesis genes and starch degradation genes, respectively, in dry seeds accelerate transcriptional regulations. However, only hypermethylation of OsAmy3D was significantly remained in developing seeds of the heat-heat plants. Since OsAmy3D is known to express in ventral side under heat stress (Nakata et al. 2017), it was expected that highly suppressed expression thereof due to the hypermethylation of its promoter might play an important role to reduce belly-white chalky grains of the heat-heat plants, resulting in overall improved quality.

On the basis of such phenotypic changes as described above, it can be considered that alterations of global DNA methylation caused by heat stress during grain filling play important roles in this phenomenon. It is known that methylation in promoter region of a gene is associated with its transcriptional regulation (Zhang et al. 2006 Cell). In the present study, DMRs were found in gene regions, and the numbers thereof were highest especially in the promoters, which might contribute to various transcriptional changes during growth. In addition, DMRs were found also in TE regions, especially in retrotransposons, which are known to respond to various stimuli and affect gene expression (Galindo-Gonzalez et al. 2017). On the basis of the results described above, the present study proposes the role of DNA methylation for transgenerational memory in rice. However, TE regulations and other epigenetic marks such as histone modification must be further elucidated in the future.

In conclusion, the present study proposes that heat stress during grain filling profoundly affects phenotypic changes of progeny plants through changes in global DNA methylation in seeds, a part of which remained at specific loci in developing organs and is involved in transcriptional regulations. Among the phenotypic changes observed, higher yield at harvest due to increase in tiller number and better grain quality due to acquired thermotolerance are considered as advantageous traits for rice production and would hugely benefit human beings.

It can be expected that what have been elucidated by the present study can be applied to sustainable agriculture using plants' own “epigenetic memory (epi-memory)” without using genetic manipulation for various plant species and various abiotic stresses. Further, the inventors of the present invention wish that the present study would shed a novel light for conducting further interesting and astonishing researches to improve world's agricultural production toward climate changes in the up-coming future.

### Wheat

Wheat (Triticum aestivum L. cv. Shiroganekomugi) was cultivated. The plants were grown as in the normal case and as in the case of rice, except that the treatment temperatures were 15, 20, and 25° C.

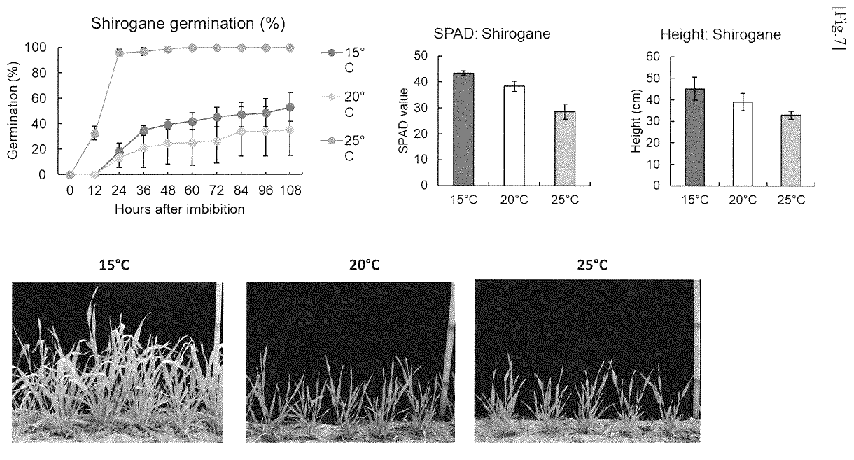

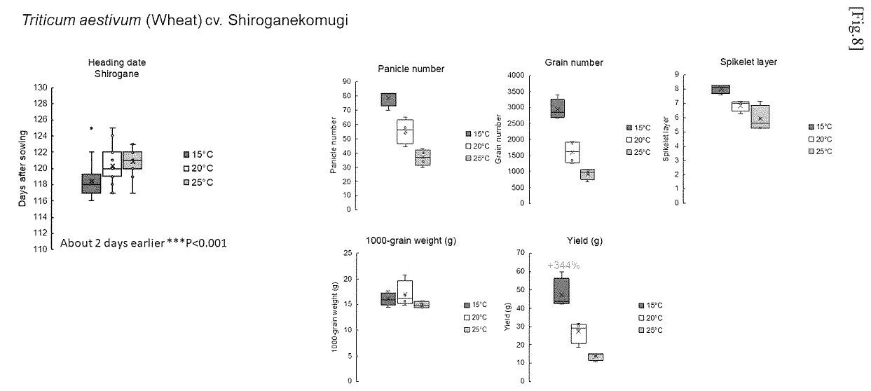

The results are shown in FIGS. 7 and 8. The same results were obtained for wheat as in the case of rice.

### Increased Biomass of Heat Memory Seeds Under Low Nitrogen Condition

### [Materials and Methods]

Germinated rice (Oryza sativa L. cv. Nipponbare, the same variety was used in the following experiments, unless especially noted) seeds were sown in sand, and dry matter was measured at the three-leaf stage. As for the fertilizer conditions, a solution containing 2.27 g/L of potassium chloride (60% potassium) and 7.77 g/L of superphosphate lime (17.5% phosphorus) was prepared with water, and 1 mL of the solution was added per individual plant at sowing, without adding nitrogen. Normally, 1 mL per plant of a 6.48 g/L of ammonium sulfate (21% nitrogen) solution is added in addition to the above solution.

### [Results]

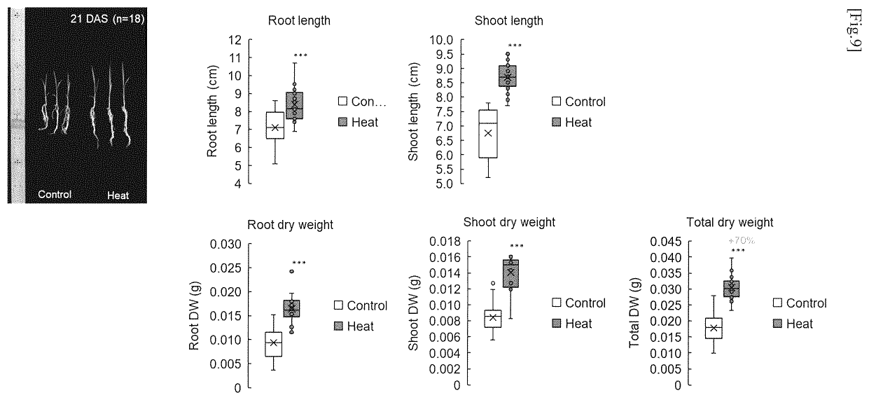

Under the low nitrogen condition, heat memory seeds (refers to seeds that were subjected to a heat stress treatment during the grain filling stage (Suriyasak et al. 2020), the term heat memory seeds refer to these seeds also in the following experiments, unless especially noted) showed increases in aboveground and belowground lengths and dry weight. The whole dry weights of the plants grown from the heat memory seeds increased by 70% compared with the control (FIG. 9).

### Acquisition of Heat Tolerance by Heat Memory Seeds Under Heat Stress

### [Materials and Methods]

Equally germinated rice seeds were sown and subjected to heat stress at 35° C. in an incubator, and the growth of germinated shoots was measured. The rice plants were also subjected to heat stress at 35° C. four weeks after the germination, and the effects of the heat were analyzed with a FLIR C2 thermal imaging camera (FLIR System AB, Sweden).

### [Results]

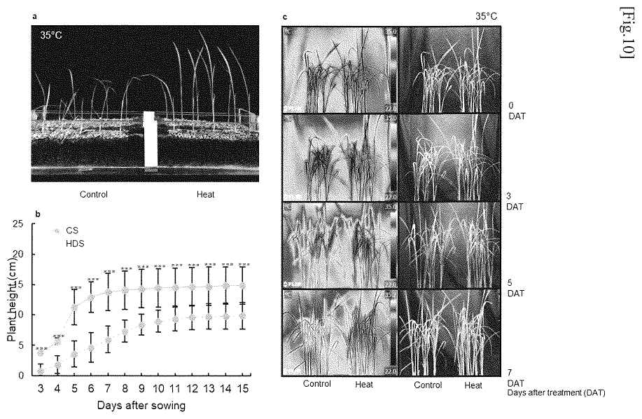

The plants grown from the heat memory seeds (HDS) could grow under a higher temperature stress condition (35° C.) and transpired a lot more compared with the control (CS), and it was revealed that they acquired stress tolerance (FIG. 10).

### Barley, Lettuce, Cowpea, and Soybean

### [Materials and Methods]

Barley (Hordeum vulgare L. cv. Ichibanboshi), which is a monocotyledon, was cultivated. The treatment temperature (for heat stress during the grain filling stage) was set at 15, 20, and 25° C. As a dicotyledon, lettuce (Lactuca sativa) was cultivated, and the heat treatment temperature was set at 25° C. for the variety Chima Sanchu and 30° C. for the variety Red Fire. Cowpea (Vigna unguiculata cv. IT98K-205-8) was cultivated with allowing determinate growth (Heat (D)) or indeterminate growth (Heat (D)) and setting the heat treatment temperature at 25° C. (Control) and 30° C. (Heat (D) and Heat (I)). For soybean (Glycine Max (L.) Merr. cv. Fukuyutaka), the heat treatment temperature was set at 25° C. and 30° C.

### [Results]

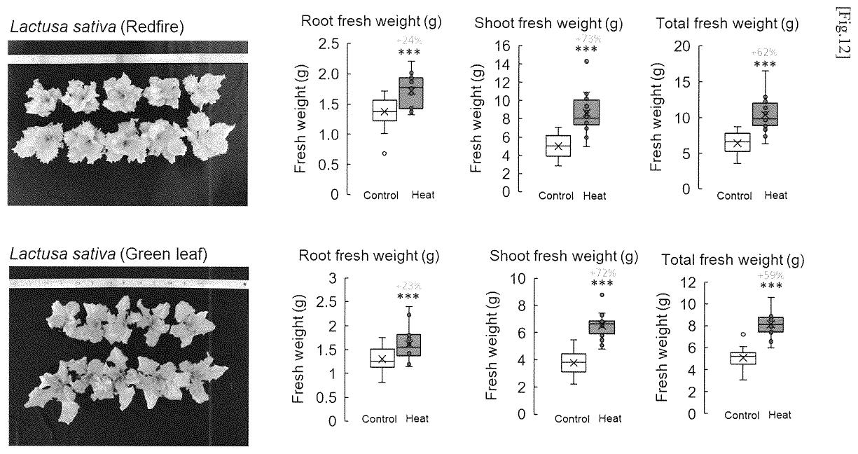

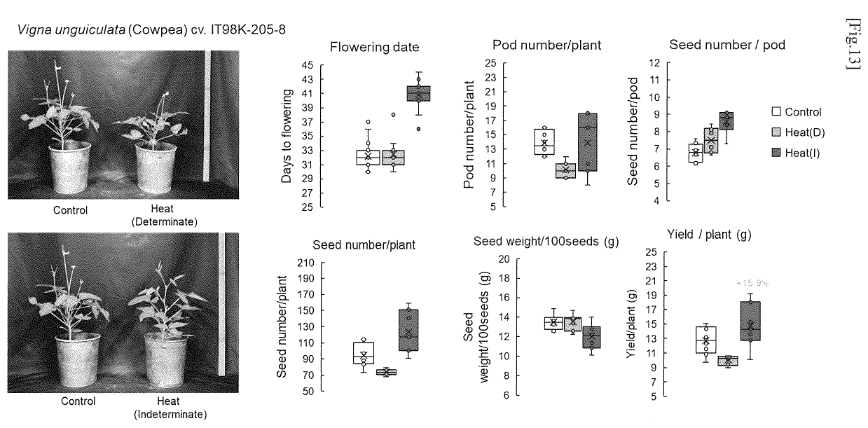

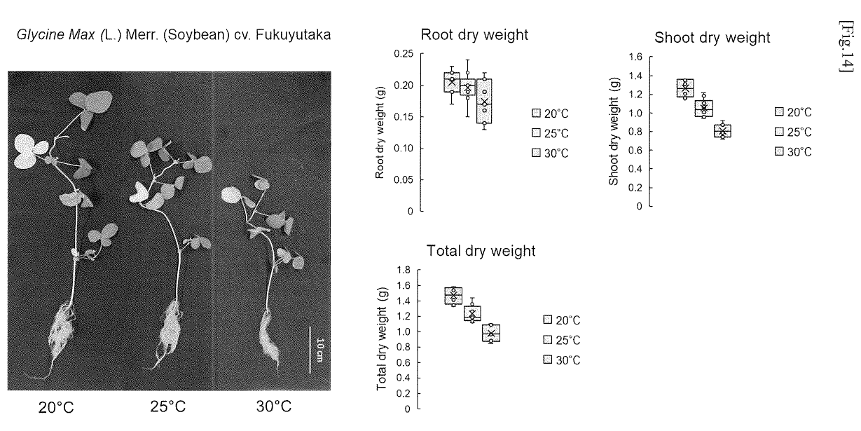

Results similar to those for rice were obtained for the monocotyledonous and dicotyledonous crops (FIGS. 12 to 14).

### References Cited in the Section of Examples

Suriyasak, C. et al. Mechanism of delayed seed germination caused by high temperature during grain filling in rice (Oryza sativa L.). Sci. Rep. 10, 17378 (2020) (Non-patent document 2 mentioned above).

Tanamachi, K. et al. Differential responses to high temperature during maturation in heat-stress-tolerant cultivars of Japonica rice. Plant Prod. Sci. 19, 300-308 (2016).

Liu, X. et al. ζ-Carotene isomerase suppresses tillering in rice through the coordinated biosynthesis of strigolactone and abscisic acid. Mol. Plant 13, 1784-1801 (2020).

Hakata, M. et al. Suppression of α-amylase genes improve quality of rice grain ripened under high temperature. Plant Biotechnol. J. 10, 1110-1117 (2012).

Suriyasak, C. et al. Reactive oxygen species induced by heat stress during grain filling of rice (Oryza sativa L.) are involved in occurrence of grain chalkiness. J. Plant Physiol. 216, 52-57 (2017).

Wang, L. & Li, J. Branching in rice. Curr. Opin. Plant Biol. 14, 94-99 (2011).

Tsuji, H. Hd3a promotes lateral branching in rice. Plant J. 82, 256-266 (2015).

Hussien, A. et al. Genetics of tillering in rice and barley. Plant Genome 7, 1-20 (2014).

Ishimaru, T. et al. Formation of grain chalkiness and change in water distribution in developing rice caryopses grown under high-temperature stress. Journal of Cereal Science, 50, 166-174.10.1016/j.jcs.2009.04.011

Zheng, X. et al. Transgenerational epimutations induced by multi-generation drought imposition mediate rice plant's adaptation to drought condition. Sci. Rep. 7, 39843 (2017)

Soliman, W. S. et al. Heat tolerance and suppression of oxidative stress: Comparative analysis of 25 cultivars of the C3 grass Lolium perenne. Environ. Exp. Bot. 78, 10-17 (2015).

Yang, Y. et al. PGL, encoding chlorophyllide a oxygenase 1, impacts leaf senescence and indirectly affects grain yield and quality in rice. J. Exp. Bot. 67, 1297-1310 (2016).

Luan, W. et al. The effect of the crosstalk between photoperiod and temperature on the heading-date in rice. PLOS One 4, e5891 (2009).

Du, A. et al. The DTH8-Hd1 module mediates day-length-dependent regulation of rice flowering. Mol. Plant 10, 948-961 (2017).

Koumoto, T. et al. Transgenerational effect of maternal growth environment on flowering date in rice (Oryza sativa L.). Environ. Exp. Bot. 155, 307-312 (2018)

Liu, X. et al. The rice enhancer of zeste [E (z)] genes SDG711 and SDG718 are respectively involved in long day and short day signaling to mediate the accurate photoperiod control of flowering time. Front. Plant Sci. 5, DOI: 10.3389/fpls.2014.0059 (2014).

Liu, J. et al. An H3K27me3 demethylase-HSFA2 regulatory loop orchestrates transgenerational thermomemory in Arabidopsis. Cell Res. 29, 379-390 (2019).

Yamakawa, H., Hirose, T., Kuroda, M. & Yamaguchi, T. Comprehensive expression profiling of rice grain filling-related genes under high temperature using DNA microarray. Plant Physiol. 144, 258-277 (2007).

Nakata, M. et al. High temperature-induced expression of rice α-amylase in developing endosperm produces chalky grains. Front. Plant Sci. 8, 2089 (2017).

Zhang, X. et al. Genome-wide-high-resolution mapping and functional analysis of DNA methylation in Arabidopsis. Cell 126, 1189-1201 (2006).

Galindo-Gonzalez, L. et al. LTR retrotransposons in plants: Engines of evolution. Gene 626, 14-25 (2017).

### Sequences Mentioned in Sequence Listing

SEQ ID NO: 1, OsSLB1:proP1
SEQ ID NO: 2, OsSLB1:proP2
SEQ ID NO: 3, OsSLB1:proP3
SEQ ID NO: 4, OsHd1:proP1
SEQ ID NO: 5, OsHd1:proP2
SEQ ID NO: 6, OsHd1:proP3
SEQ ID NO: 7, OsHd3a:proP1
SEQ ID NO: 8, OsHd3a:proP2

### SEQ ID NO: 9, OsAGPS2b:pro

### SEQ ID NO: 10, OsGBSSI:pro

### SEQ ID NO: 11, OsSuSy2:pro

### SEQ ID NO: 12, OsAmy1A:pro

### SEQ ID NO: 13, OsAmy3D:pro

### INDUSTRIAL APPLICABILITY

According to the present invention, seeds for crop production with enhanced environmental stress tolerance can be produced, which is useful in the fields of agriculture and industrial production of agricultural crops. The present invention is expected to be used for the production of high-yielding and fast-growing agricultural crops, production of seeds for production in factories, production of agricultural crops in factories, and construction of production systems for agricultural crops and seeds.

1. A seed of a plant or a product thereof, or a harvested product obtained therefrom or a processed product thereof, the seed having a promoter with an effectively controlled methylation level, the promoter being for any gene selected from the group consisting of the SLB1 gene, Hd1 gene, AGPS2b gene, GBSSI gene, SuSy2 gene, AMy1A gene, and Amy3D gene.

2. The seed or a product thereof, or a harvested product obtained therefrom or a processed product thereof according to claim 1, wherein the promoter is a hypermethylated promoter for any gene selected from the group consisting of the SLB1 gene, AGPS2b gene, GBSSI gene, and SuSy2 gene.

3. The seed or a product thereof, or a harvested product obtained therefrom or a processed product thereof according to claim 1, wherein the promoter is a hypomethylated promoter for any gene selected from the group consisting of the Hd1 gene, AMy1A gene, and Amy3D gene.

4. The seed or a product thereof, or a harvested product obtained therefrom or a processed product thereof according to claim 2, wherein the promoter is the SLB1 gene P2 promoter.

5. The seed or a product thereof, or a harvested product obtained therefrom or a processed product thereof according to claim 4, wherein the plant is a Poaceae plant, and the promoter has a relative methylation level of 1.6% input or higher.

6. A method for producing a seed of a plant having a useful characteristic, which comprises:
the step of cultivating a first generation plant under an environmental stress; and
the step of obtaining a seed with environmental stress memory from the cultivated plant or a progeny thereof

7. The production method according to claim 6, wherein the environmental stress memory is a change in methylation level of DNA in the promoter region of the Heading date 1 (Hd1) gene.

8. The production method according to claim 6, wherein the environmental stress is temperature stress or low nitrogen stress during the seed developing period.

9. The production method according to claim 6, wherein the plant is a Poaceae plant, and the useful characteristic is any selected from the group consisting of increased biomass, accelerated flowering, increased seed yield, acquired heat tolerance during grain filling, acquired low nitrogen tolerance, and early growth.

10. The production method according to claim 6, wherein the plant is a Poaceae plant, and the useful characteristic is any selected from the group consisting of increased tiller number, increased yield per unit area, earlier heading stage, and low chalky grain rate.

11. The production method according to claim 6, wherein the plant is a plant belonging to the family Poaceae, Fabaceae, or Asteraceae, and the environmental stress is temperature stress during the seed developing period.

12. The production method according to claim 6, wherein the plant is rice, the environmental stress is heat stress during the seed developing period, and the heat stress is cultivating the plant at an environmental temperature higher by 5° C. or more than the average temperature during that period for 6 weeks or longer after heading.
